(19) United States
(12) Patent Application Publication
(10) Pub. No.: US 2004/0137052 A1
(43) Pub. Date: Jul. 15, 2004

(54) Acid-containing preparations

(76) Inventors: Yoshihiro Uchiyama, Hyogo (JP); Tomohiro Yoshinari, Hyogo (JP); Makoto Fukuta, Nara (JP)

Correspondence Address: Mark Chao, Takeda Pharmaceuticals North America Inc, Intellectual Property Department, 475 Half Day Road Suite 500, Lincolnshire, IL, 60069, US

(21) Appl. No.: 10/415,753

(22) PCT Filed: Dec. 24, 2002
(86) PCT No.: PCT/JP02/13428

(30) Foreign Application Priority Data
Dec. 25, 2001 (JP) 2001-392782

(51) Int. Cl. A61K 9/20

(52) U.S. Cl. 424/465

(57) ABSTRACT

This invention provides a preparation comprising blending (1) a compound having the anti-allergy activity, the anti-histamine activity, the anti-inflammatory activity, the anti-PAF activity and/or the eosinophile chemotaxis inhibiting activity (especially, the compound is a basic compound or an amphoteric compound), and (2) an acidic compound as a preparation which is excellent in the digestive tract absorbability and the stability.

### TECHNICAL FIELD

The present invention relates to an acid-blended preparation and a process for producing the same.

### BACKGROUND ART

Among physiologically active substances, there are substances having a great fluctuation in the digestive tract absorbability. These physiologically active substances having a great fluctuation in the oral absorbability are forced to be changed into the dosage form such as an intravenous injection and an intramuscular injection due to that fluctuation, in many cases. However, the injections can not be necessarily said to be a suitable dosage form from a viewpoint of the simplicity of its use, in physiologically active substances which require repetitive administration or long term administration.

The present invention handles a difference derived from a fluctuation between individuals and a fluctuation in the same individual of in vivo factors, in particular, gastric pH, and provides a preparation of a physiologically active substance (a compound having, in particular, the anti-allergy activity, the anti-histamine activity, the anti-inflammatory activity, the anti-PAF activity and/or the eosinophile chemotaxis inhibiting activity) which is difficult to be influenced by a fluctuation in gastric pH in the living body. That is, the present invention provides a preparation comprising blending an acidic compound for the purpose of solubilizing a physiologically active substance having the pH dependent solubility.

### DISCLOSURE OF THE INVENTION

In order to solve the aforementioned problems, the present inventors intensively studied, found that a preparation comprising blending a physiologically active substance which is a basic compound or an amphoteric compound, in particular, a compound having the anti-allergy activity, the anti-histamine activity, the anti-inflammatory activity, the anti-PAF activity and/or the eosinophile chemotaxis inhibiting activity, and an acidic compound, is excellent in the absorbability and the stability and, further studied , which resulted in completion of the present invention.

That is, the present invention relates to:

[1] a preparation comprising blending (1) a compound having the anti-allergy activity, the anti-histamine activity, the anti-inflammatory activity, the anti-PAF activity and/or the eosinophile chemotaxis inhibiting activity, and (2) an acidic compound,

[2] the preparation described in the above [1], wherein the compound having the anti-allergy activity, the anti-histamine activity, the anti-inflammatory activity, the anti-PAF activity and/or the eosinophile chemotaxis inhibiting activity is a basic compound,

[3] the preparation described in the above [1], wherein the compound having the anti-allergy activity, the anti-histamine activity, the anti-inflammatory activity, the anti-PAF activity and/or the eosinophile chemotaxis inhibiting activity is an amphoteric compound,

[4] the preparation described in the above [1], wherein the solubility of the compound having the anti-allergy activity, the anti-histamine activity, the anti-inflammatory activity, the anti-PAF activity and/or the eosinophile chemotaxis inhibiting activity at pH 3 or lower is 10 times or more the solubility at pH 5 to 8,

[5] the preparation described in the above [1], wherein the compound having the anti-allergy activity, the anti-histamine activity, the anti-inflammatory activity, the anti-PAF activity and/or the eosinophile chemotaxis inhibiting activity is a compound represented by the formula:

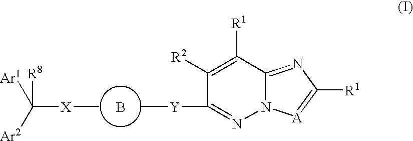

wherein Ar¹ and Ar² each is an aromatic group optionally having substituents, Ar¹ and Ar² optionally form a condensed cyclic group together with the adjacent carbon atom, ring B is a nitrogen-containing heterocycle optionally having substituents, X and Y are the same or different and each is a bond, an oxygen atom, S(O)ₚ (wherein p is an integer of 0 to 2), NR⁴ (wherein R⁴ is a hydrogen atom or a lower alkyl group) or a divalent linear lower hydrocarbon group optionally having substituents and containing 1 to 3 hetero atom(s), A is a nitrogen atom or CR⁷ (wherein R⁷ is a hydrogen atom, a halogen atom, a hydrocarbon group optionally having substituents, an acyl group or a hydroxy group optionally having substituents), R¹, R² and R³ are the same or different and each is a hydrogen atom, a halogen atom, a hydrocarbon group optionally having substituents, an acyl group or a hydroxy group optionally having substituents, R⁸ is a hydrogen atom, a hydroxy group optionally substituted by a lower alkyl group, or a carboxyl group (hereinafter, abbreviated as compound (I) in some cases) or a salt thereof,

[6] the preparation described in the above [1], wherein the compound having the anti-allergy activity, anti-histamine activity, the anti-inflammatory activity, the anti-PAF activity and/or the eosinophile chemotaxis inhibiting activity is ethyl

2-[6-[3-[4-(diphenylmethoxy)piperidino]propylamino]imidazo[1,2-b]pyridazin-2-yl]-2-methylpropionate,

2-[6-[3-[4-(diphenylmethoxy)piperidino]propylamino]imidazo[1,2-b]pyridazin-2-yl]-2-methylpropionic acid or a salt thereof,

N-[6-[3-[4-(diphenylmethoxy)piperidino]propylamino]imidazo[1,2-b]pyridazin-2-carbonyl]glycine ethyl ester or a salt thereof, ethyl

2-[6-[3-[4-(diphenylmethoxy)piperidino]propylamino]-3-methylim idazo[1,2-b]pyridazin-2-yl]-2-methylpropionate or a salt thereof, or

2-[6-[3-[4-(diphenylmethoxy)piperidino]propylamino]imidazo[1,2-b]pyridazin-2-yl]-2-methylpropionic acid dihydrate,

[7] the preparation described in the above [1], wherein the acidic compound is solid,

[8] the preparation described in the above [1], wherein 50% or more of particles constituting the acidic compound are particles of 50 μm to 1.5 mm,

[9] the preparation described in the above [1], wherein 50% or more of particles constituting the acidic compound are particles of 150 μm to 1.0 mm,

[10] the preparation described in the above [1], wherein particles of 50 μm or smaller among particles constituting the acidic compound are 20% or less of the all particles,

[11] the preparation described in the above [1], wherein the acidic compound is carboxylic acid, sulfonic acid, acidic polysaccharide or acidic amino acid,

[12] the preparation described in the above [1], wherein the acidic compound is carboxylic acid,

[13] the preparation described in the above [12], wherein carboxylic acid is fumaric acid, adipic acid, malic acid, acetic acid, tartaric acid, succinic acid or citric acid,

[14] the preparation described in the above [12], wherein carboxylic acid is tartaric acid, succinic acid or citric acid,

[15] the preparation described in the above [12], wherein carboxylic acid is citric acid,

[16] the preparation described in the above [1], which contains 0.1 to 10 parts by weight of the acidic compound relative to 1 part by weight of the compound having the anti-allergy activity, the anti-histamine activity, the anti-inflammatory activity, the anti-PAF activity and/or the eosinophile chemotaxis inhibiting activity,

[17] the preparation described in the above [1], which is a tablet,

[18] the preparation described in the above [1], which comprises blending a granule containing-the compound having the anti-allergy activity, the anti-histamine activity, the anti-inflammatory activity, the anti-PAF activity and/or the eosinophile chemotaxis inhibiting activity and a granule containing the acidic compound,

[19] the preparation described in the above [18], wherein the granule containing the compound having the anti-allergy activity, the anti-histamine activity, the anti-inflammatory activity, the anti-PAF activity and/or the eosinophile chemotaxis inhibiting activity contains 50% or more particles of 50 μm to 1.5 mm, and the granule containing the acidic compound contains 50% or more of particles of 50 μm to 1.5 mm,

[20] the preparation described in the above [18], wherein the granule containing the compound having the anti-allergy activity, the anti-histamine activity, the anti-inflammatory activity, the anti-PAF activity and/or the eosinophile chemotaxis inhibiting activity contains 50% or more particles of 150 μm to 1.0 mm, and the granule containing the acidic compound contains 50% or more of particles of 150 μm to 1.0 mm,

[21] the preparation described in the above [1], which is a multi-layered tablet,

[22] the preparation described in the above [17] or the above [21], which is a coated preparation,

[23] the preparation described in the above [1], wherein talc and/or magnesium stearate is (are) further added thereto,

[24] a process for preparing the preparation as defined in the above [1], which comprises incorporating a granule containing a compound having the anti-allergy activity, the anti-histamine activity, the anti-inflammatory activity, the anti-PAF activity and/or the eosinophile chemotaxis inhibiting activity and a granule containing an acidic compound.

### BRIEF DESCRIPTION OF THE DRAWINGS

FIG. 1 is a drawing of dissolution change of compound A toward pH. The vertical line shows solubility of compound A (mg/mL) and the horizontal axis shows pH.

FIG. 2 is a drawing which shows the result of dissolution test of the tablet containing compound A (25 mg) produced in Example 4 (n=3, mean ±SD). —▴— shows the solubility of the pre-reserved tablet. —♦— shows the solubility of the tablet reserved under the condition adjusted at 25° C. and 60% RH for 1 month. —▪— shows the solubility of the tablet reserved under condition adjusted at 40° C. and 75% RH for 1 month. The vertical line shows dissolution rate (%) and the horizontal axis shows time (min).

FIG. 3 is a drawing which shows the result of dissolution test of the tablet containing compound A (100 mg) produced in Example 4 (n=3, mean ±SD). —▴— shows the solubility of the pre-reserved tablet. —♦— shows the solubility of the tablet reserved under the condition adjusted at 25° C. and 60% RH for 1 month. —▪— shows the solubility of the tablet reserved under condition adjusted at 40° C. and 75% RH for 1 month. —X— shows the solubility of the tablet reserved under condition adjusted at 40° C. and 11% RH for 1 month. —◯— shows the solubility of the tablet reserved under condition adjusted at 40° C. and 33% RH for 1 month. The vertical line shows dissolution rate (%) and the horizontal axis shows time (min).

FIG. 4 is a drawing which shows the result of dissolution test of the tablet containing compound A (12.5 mg) produced in Example 5 (n=6, mean ±SD). —♦— shows the solubility of the pre-reserved tablet. —▪— shows the solubility of the tablet reserved under the condition adjusted at 40° C. and 75% RH for 1 month. The vertical line shows dissolution rate (%) and the horizontal axis shows time (min).

FIG. 5 is a drawing which shows the result of dissolution test of the tablet containing compound A (100 mg) produced in Example 5 (n=6, mean ±SD). —♦— shows the solubility of the pre-reserved tablet. —▪— shows the solubility of the tablet reserved under the condition adjusted at 40° C. and 75% RH for 1 month. The vertical line shows dissolution rate (%) and the horizontal axis shows time (min).

FIG. 6 is a drawing which shows the result of dissolution test of the tablet containing compound A (12.5 mg) produced in Example 6 (n=6, mean ±SD). —♦— shows the solubility of the pre-reserved tablet. —▪— shows the solubility of the tablet reserved under the condition adjusted at 40° C. and 75% RH for 1 month. The vertical line shows dissolution rate (%) and the horizontal axis shows time (min).

FIG. 7 is a drawing which shows the result of dissolution test of the tablet containing compound A (25 mg) produced in Example 6 (n=6, mean). —♦— shows the solubility of the pre-reserved tablet. —♦— shows the solubility of the tablet reserved under the condition adjusted at 40° C. and 75% RH for 1 month. The vertical line shows dissolution rate (%) and the horizontal axis shows time (min).

FIG. 8 is a drawing which shows the result of dissolution test of the tablet containing compound A (50 mg) produced in Example 6 (n=6, mean). —♦— shows the solubility of the pre-reserved tablet. —▪— shows the solubility of the tablet reserved under the condition adjusted at 40° C. and 75% RH for 1 month. The vertical line shows dissolution rate (%) and the horizontal axis shows time (min).

“A compound having anti-allergy activity, anti-histamine activity, anti-inflammatory activity, anti-PAF activity and/or eosinophile chemotaxis inhibiting activity” used in this invention is, for example, a non-peptide compound whose molecular weight is less than 1000, preferably less than 900, more preferably less than 800.

As “a compound having anti-allergy activity, anti-histamine activity, anti-inflammatory activity, anti-PAF activity and/or eosinophile chemotaxis inhibiting activity” used in this invention, a basic compound or an amphoteric compound is preferably used.

In this specification, “a basic compound” and “an amphoteric compound” stand for the compound which is water-soluble in the acidic condition and water-insoluble in the neutral condition. The term “water-insoluble” used herewith means that the solubility of the compound in water at 25° C. is less than 1,000 ppm (10 mg/mL) (preferably less than 10 ppm (0.1 mg/mL)). The solubility is measured by the conventional method.

And the term “a basic compound” and “an amphoteric compound” can be also defined by using the value of the pKa (the logarithm of inverse of the ionization constant of an acid) in the partial structure of the compound. That is, “a basic compound” is a compound having a partial structure whose pKa is more than 7.5, preferably, more than 8.5. And “an amphoteric compound” is a compound having a partial structure whose pKa is more than 7.5 and a partial structure whose pKa is less than 6.5, preferably a compound having a partial structure whose pKa is more than 8.5 and a partial structure whose pKa is less than 5.5.

The preferable “a basic compound” and “an amphoteric compound” in this specification is, for example, a compound whose solubility in less than pH 3 is more than 10 times than that in pH 5 to 8, preferably a compound whose solubility in less than pH 3 is more than 30 times than that in pH 5 to 8, more preferably a compound whose solubility in less than pH 3 is more than 100 times than that in pH 5 to 8, and especially, a compound whose solubility in less than pH 3 is more than 1,000 times than that in pH 5 to 8.

“The compound having anti-allergy activity, anti-histamine activity, anti-inflammatory activity, anti-PAF (platelet-aggregating factor) activity, eosinophile chemotaxis inhibiting activity” used in this invention is, for example, diphenhydramine, clemastine fumarate, dimenhydrinate, chlorpheniramine maleate, triprolidine hydrochloride, promethazine hydrochloride, alimemazine tartrate, isothipendyl hydrochloride, homochlorcyclizine hydrochloride, hydroxyzine, cyproheptadine hydrochloride, mequitazine, terfenadine, epinastine hydrochloride, astemizole, ebastine, cetirizine hydrochloride, sodium crmoglicate, tranilast, ketotifen fumarate, azelastine hydrochloride, oxatomide, amlexanox, repirinast, ibudilast, pemirolast, tazanolast, ozagrel hydrochloride, suplatast tosilate, seratrodast, emedastine difumarate, pranlukast hydrate and the compound (I) described above or salt thereof and the like. Among them, the compound (I) or salt thereof and the like is preferable.

In the above-mentioned formulas, Ar¹ and Ar² are each an “aromatic group optionally having substituents”, and Ar¹ and Ar² may form a condensed cyclic group together with the adjacent carbon atom.

As the “aromatic group” represented by Ar¹ and Ar^{2,} for example,

(1) a monocyclic or condensed polycyclic aromatic hydrocarbon group, more specifically a 6 to 14-membered monocyclic or condensed polycyclic aromatic hydrocarbon group exemplified by C₆₋₁₄ aryl group such as phenyl, tolyl, xylyl, biphenyl, 1-naphthyl, 2-naphthyl, 2-indenyl, 1-anthryl, 2-anthryl, 9-anthryl, 1-phenanthryl, 2-phenanthryl, 3-phenanthryl, 4-phenanthryl, 9-phenanthryl and the like, and the like (preferably phenyl, tolyl, xylyl, biphenyl, 1-naphthyl, 2-naphthyl and the like, particularly preferably phenyl and the like), and the like, and

(2) a monocyclic group (preferably 5 to 8-membered) containing, other than carbon atom, preferably 1 or 2 kinds of 1 or more (e.g., 1 to 4, preferably 1 to 3) heteroatoms selected from nitrogen atom, sulfur atom and oxygen atom, or a condensed aromatic heterocyclic group thereof, more specifically aromatic heterocycle such as thiophene, benzo[b]thiophene, benzo[b]furan, benzimidazole, benzoxazole, benzothiazole, benzisothiazole, naphtho[2,3-b]thiophene, thianthrene, furan, isoindolizine, oxanthrene, phenoxathiin, pyrrole, imidazole, triazole, thiazole, oxazole, pyrazole, pyridine, pyrazine, pyrimidine, pyridazine, indole, isoindole, 1H-indazole, purine, 4H-quinolizine, isoquinoline, quinoline, phthalazine, naphthyridine, quinoxaline, quinazoline, cinnoline, carbazole, β-carboline, phenanthridine, acridine, phenazine, isothiazole, phonothiazine, isoxazole, furazan, phenoxazine or isochroman and the like (preferably pyridine, thiophene or furan and the like, more preferably pyridine and the like), or

(3) a monovalent group obtained by removing an optional hydrogen atom from a ring formed by condensing these rings (preferably the aforementioned monocyclic heterocycle) with 1 or plural (preferably 1 or 2, more preferably 1) aromatic rings (e.g., the above-mentioned aromatic hydrocarbon group and the like, preferably benzene ring and the like).

As the “aromatic group” of the “aromatic group optionally having substituents” represented by Ar¹ and Ar², for example, phenyl group and the like are preferable.

As the “substituent” of the aromatic group represented by Ar¹ and Ar², for example, (i) halogen atom (e.g., fluorine, chlorine, bromine, iodine), (ii) lower alkylenedioxy group (e.g., C₁₋₃ alkylenedioxy group such as methylenedioxy, ethylenedioxy and the like, and the like), (iii) nitro group, (iv) cyano group, (v) optionally halogenated lower alkyl group, (vi) optionally halogenated lower alkenyl group, (vii) optionally halogenated lower alkynyl group, (viii) lower cycloalkyl group (e.g., C₃₋₆ cycloalkyl group such as cyclopropyl, cyclobutyl, cyclopentyl, cyclohexyl and the like, and the like), (ix) optionally substituted lower alkoxy group, (x) optionally halogenated lower alkylthio group, (xi) hydroxy group, (xii) amino group, (xiii) mono-lower alkylamino group (e.g., mono-C₁₋₆ alkylamino group such as methylamino, ethylamino, propylamino, isopropylamino, butylamino and the like, and the like), (xiv) di-lower alkylamino group (e.g., di-C₁₋₆ alkylamino group such as dimethylamino, diethylamino, dipropylamino, dibutylamino and the like, and the like), (xv) 5 or 6-membered cyclic amino group (e.g., morpholino, piperazin-1-yl, piperidino, pyrrolidin-1-yl and the like), (xvi) lower alkyl-carbonyl group (e.g., C₁₋₆ alkyl-carbonyl group such as acetyl, propionyl and the like, and the like), (xvii) carboxyl group, (xviii) lower alkoxy-carbonyl group (e.g., C₁₋₆ alkoxy-carbonyl group such as methoxycarbonyl, ethoxycarbonyl, propoxycarbonyl, butoxycarbonyl and the like, and the like), (xix) carbamoyl group, (xx) thiocarbamoyl, (xxi) mono-lower alkyl-carbamoyl group (e.g., mono-C₁₋₆ alkyl-carbamoyl group such as methylcarbamoyl, ethylcarbamoyl and the like, and the like), (xxii) di-lower alkyl-carbamoyl group (e.g., di-C₁₋₆ alkylcarbamoyl group such as dimethylcarbamoyl, diethylcarbamoyl and the like, and the like), (xxiii) aryl-carbamoyl (e.g., C₆₋₁₀ aryl-carbamoyl such as phenylcarbamoyl, naphthylcarbamoyl and the like, and the like), (xxiv) sulfo group, (xxv) lower alkylsulfonyl group (e.g., C₁₋₆ alkylsulfonyl group such as methylsulfonyl, ethylsulfonyl and the like, and the like), (xxvi) aryl group (e.g., C₆₋₁₀ aryl group such as phenyl, naphthyl and the like, and the like), (xxvii) aryloxy group (e.g., C₆₋₁₀ aryloxy group such as phenoxy, naphthyloxy and the like, and the like), (xxviii) aralkyloxy group (e.g., C₇₋₁₆ aralkyloxy group such as benzyloxy and the like, and the like) and the like are used.

As the above-mentioned “optionally halogenated lower alkyl group”, for example, lower alkyl group such as C₁₋₆ alkyl group (e.g., methyl, ethyl, propyl, isopropyl, butyl, isobutyl, sec-butyl, tert-butyl, pentyl, hexyl and the like, and the like) optionally having 1 to 3 halogen atoms (e.g., fluorine, chlorine, bromine, iodine), and the like are mentioned. Specific examples thereof include methyl, fluoromethyl, chloromethyl, difluoromethyl, trichloromethyl, trifluoromethyl, ethyl, 2-bromoethyl, 2,2,2-trifluoroethyl, propyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl, 6,6,6-trifluorohexyl and the like.

As the above-mentioned “optonally halogenated lower alkenyl group” and “optionally halogenated lower alkynyl group”, for example, lower alkenyl group (e.g., C₂₋₆ alkenyl group such as vinyl, propenyl, isopropenyl, 2-buten-1-yl, 4-penten-1-yl, 5-hexen-1-yl, and the like, and the like) optionally having 1 to 3 halogen atoms (e.g., fluorine, chlorine, bromine, iodine), and lower alkynyl group (e.g., C₂₋₆ alkynyl group such as 2-butyn-1-yl, 4-pentyn-1-yl, 5-hexyn-1-yl, and the like, and the like), optionally having 1 to 3 halogen atoms (e.g., fluorine, chlorine, bromine, iodine), and the like are used.

As the above-mentioned “optionally substituted lower alkoxy group”, for example, lower alkoxy group (e.g., C₁₋₆ alkoxy group such as methoxy, ethoxy, propoxy, isopropoxy, n-butoxy, isobutoxy, sec-butoxy, tert-butoxy and the like, and the like) optionally having 1 to 3 halogen atoms (e.g., fluorine, chlorine, bromine, iodine), mono- or di-lower alkylamino group (e.g., mono- or di-C₁₋₆ alkylamino group such as methylamino, dimethylamino, ethylamino, diethylamino and the like, and the like) or lower alkoxy-carbonyl group (e.g., C₁₋₆ alkoxy-carbonyl group such as methoxycarbonyl, ethoxycarbonyl and the like, and the like), and the like are used.

As the above-mentioned “optionally halogenated lower alkylthio group”, for example, lower alkylthio group (e.g., C₁₋₆ alkylthio group such as methylthio, ethylthio, n-propylthio, isopropylthio, n-butylthio, isobutylthio, sec-butylthio, tert-butylthio and the like, and the like) optionally having 1 to 3 halogen atoms (e.g., fluorine, chlorine, bromine, iodine), and the like are mentioned. Specific examples thereof include methylthio, difluoromethylthio, trifluoromethylthio, ethylthio, propylthio, isopropylthio, butylthio, 4,4,4-trifluorobutylthio, pentylthio, hexylthio and the like.

Specific examples of the condensed cyclic group formed by Ar¹ and Ar² together with the adjacent carbon atom include condensed cyclic group represented by

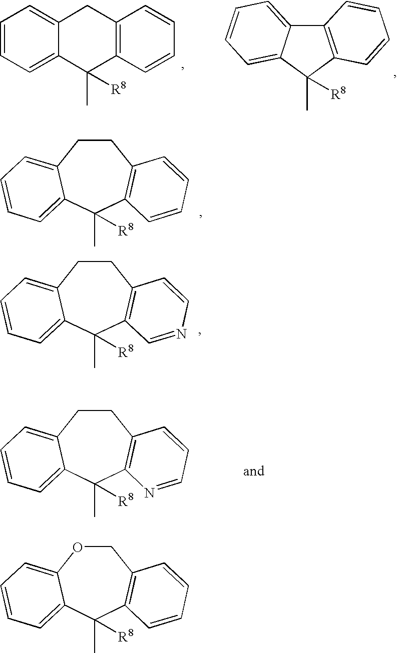

wherein R⁸ is as defined above, and the like.

Ar¹ and Ar² are the same or different and each is preferably an aromatic hydrocarbon group optionally having substituents, such as a C₆₋₁₄ aromatic hydrocarbon group, more preferably a phenyl group optionally having substituents. More specifically, Ar¹ and Ar² are each preferably (1) a phenyl group optionally substituted by halogen atom or C₁₋₆ alkyl, (2) a 5 to 8-membered aromatic heterocyclic group containing, other than carbon atom, 1 to 4 heteroatoms selected from nitrogen atom, sulfur atom and oxygen atom, and the like.

In the above-mentioned formulas, ring B is a “nitrogen-containing heterocycle optionally having substituents”.

As the “nitrogen-containing heterocycle” represented by ring B, for example, a 3 to 13-membered nitrogen-containing heterocycle containing 1 nitrogen atom, and optionally containing 1 to 3 heteroatoms selected from, for example, nitrogen atom, oxygen atom, sulfur atom and the like, and the like are used. In the above-mentioned formulas, it is preferable to form a divalent group by removing one hydrogen atom each from nitrogen atom and the other atom of ring B. Specifically, for example, 3 to 9-membered (more preferably 3 to 6-membered) nitrogen-containing heterocyclic group of

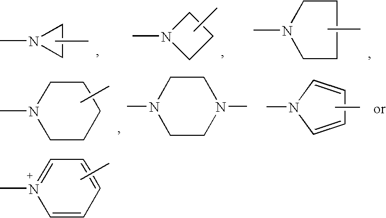

and the like, are preferable.

As the substituent of the nitrogen-containing heterocycle represented by ring B, for example, the “substituent” of the above-mentioned “aromatic group optionally having substituents” represented by Ar¹ and Ar², oxo group and the like are used.

Preferable examples of ring B include, for example, a ring represented by the formula

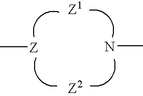

wherein Z is a nitrogen atom or a methine group, and Z¹ and Z² are each a linear C₁₋₄ alkylene group optionally substituted by hydroxy group, oxo group or C₁₋₆ alkyl group, and the like.

As the “C₁₋₆ alkyl group”, for example, linear or branched C₁₋₆ alkyl group such as methyl, ethyl, propyl, isopropyl, butyl, isobutyl, sec-butyl, tert-butyl, pentyl, hexyl and the like, and the like are used.

As the “linear C₁₋₄ alkylene group”, for example, linear C₁₋₄ alkylene group represented by methylene, ethylene, propylene and butylene are used.

As the “linear C₁₋₄ alkylene group optionally substituted by hydroxy group, oxo group or C₁₋₆ alkyl group” which is represented by Z¹ and Z², an unsubstituted linear C₁₋₄ alkylene group and the like are preferably used, and an unsubstituted linear C₁₋₂ alkylene group is particularly preferable.

As ring B, piperidine, piperazine and the like are more preferably used.

In the above-mentioned formulas, X and Y are the same or different and each is (1) a bond, (2) an oxygen atom, (3) S(O)ₚ (p is an integer of 0 to 2), (4) NR⁴ (R⁴ is a hydrogen atom or a lower alkyl group) or (5) a divalent linear lower hydrocarbon group optionally having substituents and containing 1 to 3 heteroatoms.

As the lower alkyl group represented by R⁴, for example, linear or branched C₁₋₆ alkyl group such as methyl, ethyl, propyl, isopropyl, butyl, isobutyl, sec-butyl, tert-butyl, pentyl, hexyl and the like, and the like are used.

The “divalent linear lower hydrocarbon group optionally containing 1 to 3 heteroatoms” represented by X and Y, is a group obtained by removing one hydrogen atom bonded to each of the same or different carbon atoms of lower (C₁₋₆) hydrocarbon, namely 2 hydrogen atoms, which is, for example, a group optionally having a heteroatom selected from oxygen atom, NR^{4′} (R^{4′} is a hydrogen atom or a lower alkyl group), sulfur atom and the like, in a hydrocarbon chain.

As the lower alkyl group represented by R^{4′}, for example, linear or branched C₁₋₆ alkyl group such as methyl, ethyl, propyl, isopropyl, butyl, isobutyl, sec-butyl, tert-butyl, pentyl, hexyl and the like, and the like are used.

Specific examples of the “divalent linear lower hydrocarbon group” include

(i) C₁₋₆ alkylene group (e.g., —CH₂—, —(CH₂)₂—, —(CH₂)₃—, —(CH₂)₄—, —(CH₂)₅—, —(CH₂)₆— and the like),

(ii) C₂₋₆ alkenylene group (e.g., —CH═CH—, —CH═CH—CH₂—, —CH₂—CH═CH—CH₂—, —(CH₂)₂—CH═CH—CH₂—, —(CH₂)₂—CH═CH—(CH₂)₂—, —(CH₂)₃—CH═CH—CH₂— and the like),

(iii) C₂₋₆ alkynylene group (e.g., —C≡C—, —C═C—CH₂—, —CH₂—C≡C—CH₂—, —(CH₂)₂—C≡C—CH₂—, —(CH₂)₂—C≡C—(CH₂)₂—, —(CH₂)₃—C≡C—CH₂— and the like) and the like.

As the “substituent” of the “divalent linear lower hydrocarbon group optionally containing 1 to 3 heteroatoms” represented by X and Y, for example, the “substituent” of the above-mentioned “aromatic group optionally having substituents” represented by Ar¹ and Ar², oxo group and the like are used. Particularly, hydroxy group and oxo group are preferable.

As X, a bond, an oxygen atom or NH is preferable, and particularly, a bond or an oxygen atom is preferable.

As Y, preferred is, for example, a group of the formula

—(CH₂)ₘ—Y¹—(CH₂)ₙ—Y²—

wherein Y¹ and Y² are the same or different and each is a bond, an oxygen atom, S(O)ₚ (p is as defined above), NR^{4′} (R^{4′} is as defined above), a carbonyl group, a carbonyloxy group or a group of the formula

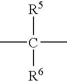

wherein R⁵ and R⁶ are the same or different and each is a hydroxy group or a C₁₋₄ alkyl group, and m and n are each an integer of 0 to 4 (provided that the sum of m and n is not more than 6, and the like.

As the “C₁₋₄ alkyl group” represented by R⁵ and R⁶, for example, linear or branched C₁₋₄ alkyl group such as methyl, ethyl, propyl, isopropyl, butyl, isobutyl, sec-butyl, tert-butyl and the like, and the like are used.

As Y, for example, a group represented by (i) C₁₋₆ alkylene group, (ii) —(CH₂)ₚ₁O—, (iii) —(CH₂)ₚ₁NH—, (iv) —(CH₂)ₚ₁S—, (v) —(CH₂)_{q1}CH(OH)(CH₂)_{q2}O—, (vi) —(CH₂)_{q1}CH(OH)(CH₂)_{q2}NH—, (vii) —(CH₂)_{q1}CH(OH)(CH₂)_{q2}S—, (viii) —(CH₂)ₚ₁CONH—, (ix) —COO(CH₂)ₚ₁O—, (x) —COO(CH₂)ₚ₁NH—, (xi) —COO(CH₂)ₚ₁S—, (xii) —(CH₂)_{q1}O(CH₂)_{q2}O—, (xiii) —(CH₂)_{q1}O(CH₂)_{q2}NH— or (xiv) —(CH₂)_{q1}O(CH₂)_{q2}S— (p¹ is an integer of 1 to 6 and q¹ and q² are each an integer of 1 to 3) is preferable.

Of these, for example, Y is preferably a bond, —(CH₂)₂—O—, —(CH₂)₃—O—, —(CH₂)₄—O—, —(CH₂)₆—O—, —(CH₂)₂—NH—, —(CH₂)₃—NH—, —(CH₂)₄—NH—, —(CH₂)₃—S—, —CH₂—CH(OH)—CH₂—O—, —(CH₂)₂—CO—NH—, —CH₂—CO—NH—, —CO—O—(CH₂)₂—O—, —CO—O—(CH₂)₃—O—, —(CH₂)₆—NH—, —(CH₂)₆—S—, —(CH₂)₂—O—(CH₂)₂—O—, —(CH₂)₂—O—(CH₂)₂—S— and the like.

In the above-mentioned formulas, A is a nitrogen atom or CR⁷ (R⁷ is a hydrogen atom, a halogen atom, a hydrocarbon group optionally having substituents, an acyl group or a hydroxy group optionally having substituents).

As the “halogen atom” represented by R⁷, fluorine, chlorine, bromine and iodine are exemplified.

The “hydrocarbon group” represented by R⁷ is, for example, a group obtained by removing one hydrogen atom from a hydrocarbon compound. Examples thereof include chain or cyclic hydrocarbon group such as alkyl group, alkenyl group, alkynyl group, cycloalkyl group, aryl group, aralkyl group and the like. Of these, chain (linear or branched) or cyclic hydrocarbon group having 1 to 16 carbon atoms and the like are preferable, and

a) alkyl group [preferably lower alkyl group (e.g., C₁₋₆ alkyl group such as methyl, ethyl, propyl, isopropyl, butyl, isobutyl, sec-butyl, tert-butyl, pentyl, hexyl and the like, and the like)],

b) alkenyl group [preferably lower alkenyl group (e.g., C₂₋₆ alkenyl group such as vinyl, allyl, isopropenyl, butenyl, isobutenyl, sec-butenyl and the like, and the like)],

c) alkynyl group [preferably lower alkynyl group (e.g., C₂₋₆ alkynyl group such as propargyl, ethynyl, butynyl, 1-hexynyl and the like, and the like)],

d) cycloalkyl group [preferably lower cycloalkyl group (e.g., C₃₋₆ cycloalkyl group such as cyclopropyl, cyclobutyl, cyclopentyl, cyclohexyl optionally condensed with benzene ring optionally having 1 to 3 lower alkoxy groups (e.g., C₁₋₆ alkoxy group such as methoxy and the like, and the like), and the like, and the like)],

e) aryl group (e.g., C₆₋₁₄ aryl group such as phenyl, tolyl, xylyl, biphenyl, 1-naphthyl, 2-naphthyl, 2-indenyl, 1-anthryl, 2-anthryl, 9-anthryl, 1-phenanthryl, 2-phenanthryl, 3-phenanthryl, 4-phenanthryl, 9-phenanthryl and the like, and the like, preferably phenyl group),

f) aralkyl group [preferably lower aralkyl group (e.g., C₇₋₁₆ aralkyl group such as benzyl, phenethyl, diphenylmethyl, 1-naphthylmethyl, 2-naphthylmethyl, 2-phenylethyl, 2-diphenylethyl, 1-phenylpropyl, 2-phenylpropyl, 3-phenylpropyl, 4-phenylbutyl, 5-phenylpentyl and the like, and the like, more preferably benzyl group)] and the like are preferable.

As the “substituent” of the “hydrocarbon group” represented by R⁷, for example, the “substituent” of the above-mentioned “aromatic group optionally having substituents” represented by Ar¹ and Ar², oxo group and the like are used.

As the “acyl group” represented by R⁷, for example, —(C═O)—R⁹, —SO₂—R⁹, —SO—R⁹, —(C═O)NR¹⁰R⁹, —(C═O)O—R⁹, —(C═S)O—R⁹, —(C═S)NR¹⁰R⁹, (R⁹ is a hydrogen atom, a hydrocarbon group optionally having substituents or a hydroxy group optionally having substituents, and R¹⁰ is a hydrogen atom or a lower alkyl group (e.g., C₁₋₆ alkyl group such as methyl, ethyl, propyl, isopropyl, butyl, isobutyl, sec-butyl, tert-butyl, pentyl, hexyl and the like, and the like, particularly C₁₋₃ alkyl group such as methyl, ethyl, propyl, isopropyl and the like, and the like are preferable)) and the like are mentioned.

Of these, preferred are —(C═O)—R⁹, —SO₂—R⁹, —SO—R⁹, —(C═O)NR¹⁰R⁹ and —(C═O)O—R⁹, and —(C═O)—R⁹ is more preferable.

The “hydrocarbon group” represented by R⁹ is a group obtained by removing one hydrogen atom from a hydrocarbon compound. Examples thereof include chain (linear or branched) or cyclic hydrocarbon group such as alkyl group, alkenyl group, alkynyl group, cycloalkyl group, aryl group, aralkyl group and the like. Specific example thereof is the above-mentioned “hydrocarbon group” represented by R⁷ and the like, and of these, chain or cyclic hydrocarbon group having 1 to 16 carbon atoms and the like are preferable, and lower (C₁₋₆) alkyl group is particularly preferable.

As the “substituent” that the “hydrocarbon group” represented by R⁹ may have, for example, the “substituent” of the above-mentioned “aromatic group optionally having substituents” represented by Ar¹ and Ar², oxo group and the like are used.

As the “hydroxy group optionally having substituents” represented by R⁹, for example, those similar to the “hydroxy group optionally having substituents” represented by R⁷ to be mentioned later and the like are used.

As the “hydroxy group optionally having substituents” represented by R⁷, for example, (1) a hydroxy group or (2) a hydroxy group having, for example, one aforementioned “hydrocarbon group optionally having substituents” and the like, instead of hydrogen atom of hydroxy group is used.

As R⁷, (1) a hydrogen atom, (2) halogen atom, (3) a C₁₋₆ alkyl group optionally substituted by carboxyl group or C₁₋₆ alkoxy-carboxyl, (4) a C₁₋₆ alkoxy group, (5) a C₁₋₆ alkoxy-carbonyl group or (6) a carboxyl group is preferable, and particularly, a hydrogen atom, a halogen atom, a C₁₋₆ alkyl group, a C₁₋₆ alkoxy-carbonyl group and a carboxyl group are preferable.

As A, a nitrogen atom or CR^{7′} (R^{7′} is a hydrogen atom, a halogen atom, a C₁₋₆ alkyl group, a C₁₋₆ alkoxy-carbonyl group or a carboxyl group) is preferable, and of these, nitrogen atom, CH and C—CH₃ are preferable, and nitrogen atom and CH are particularly preferable.

In the above-mentioned formulas, R¹, R² and R³ are the same or different and each is a hydrogen atom, a halogen atom, a hydrocarbon group optionally having substituents, an acyl group or a hydroxy group optionally having substituents.

As the “halogen atom” represented by R¹, R² and R³, fluorine, chlorine, bromine and iodine are exemplified.

As the “hydrocarbon group optionally having substituents” represented by R¹, R² and R³, for example, the above-mentioned “hydrocarbon group optionally having substituents” represented by R⁷, and the like are used.

As the “acyl group” represented by R¹, R² and R³, for example, the above-mentioned “acyl group” represented by R⁷, and the like are used.

As the “hydroxy group optionally having substituents” represented by R¹, R² and R³, for example, the above-mentioned “hydroxy group optionally having substituents” represented by R⁷, and the like are used.

R², R² and R³ are the same or different and each is (1) a hydrogen atom, (2) a C₁₋₆ alkyl group optionally substituted by carboxyl group or C₁₋₆ alkoxy-carbonyl, (3) a C₁₋₆ alkoxy group, (4) a C₁₋₆ alkoxy-carbonyl group, (5) a carboxyl group or (6) a C₆₋₁₄ aryl group (particularly phenyl) is preferable, and (1) a hydrogen atom, (2) a C₁₋₆ alkyl group optionally substituted by carboxyl group and C₁₋₆ alkoxy-carbonyl, (3) a C₁₋₆ alkoxy group, (4) a C₁₋₆ alkoxy-carbonyl group or (5) a carboxyl group are more preferable.

As R¹, (1) a hydrogen atom, (2) a C₁₋₆ alkyl group optionally substituted by a group selected from the group consisting of (i) carboxyl, (ii) C₁₋₆ alkoxy-carbonyl, (iii) hydroxy or (iv) carbamoyl optionally having mono- or di-C₁₋₆ alkyl, (3) a C₆₋₁₄ aryl group, (4) a C₁₋₆ alkoxy group, (5) a C₁₋₆ alkoxy-carbonyl group, (6) a carboxyl group, (7) a carbamoyl group optionally having C₁₋₆ alkyl optionally substituted by carboxyl or C₁₋₆ alkoxy-carbonyl, or (8) a C₃₋₆ cycloalkyl group optionally substituted by C₁₋₆ alkoxy-carbonyl and the like are also preferable.

As R², a hydrogen atom, a C₁₋₆ alkyl group, a C₁₋₆ alkoxy-carbonyl group or a carboxyl group and the like are also preferable.

As R³, a hydrogen atom is preferable.

In the above-mentioned formulas, R⁸ is a hydrogen atom, hydroxy group optionally substituted by lower alkyl group, or carboxyl group.

In the above-mentioned the formulas, the “lower alkyl group” of “hydroxy group optionally substituted by lower alkyl group”, which is represented by R⁸ is, for example, C₁₋₆ alkyl group such as methyl, ethyl, propyl, isopropyl, butyl, isobutyl, sec-butyl, tert-butyl, pentyl, hexyl and the like, and the like.

As R⁸, a hydrogen atom or a hydroxy group is preferable, and a hydrogen atom is particularly preferable.

As compound (I) of the present invention, a compound wherein Ar¹ and Ar² are each (1) a phenyl group optionally substituted by halogen atom or C₁₋₆ alkyl or (2) a 5 to 8-membered aromatic heterocyclic group containing, other than carbon atom, 1 to 4 heteroatoms selected from nitrogen atom, sulfur atom and oxygen atom, ring B is a ring represented by the formula

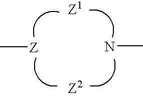

wherein Z is a nitrogen atom or a methine group, and Z¹ and Z² are each a linear C₁₋₄ alkylene group optionally substituted by hydroxy group, oxo group or C₁₋₆ alkyl group, X is a bond, an oxygen atom or NH, Y is a group represented by (i) C₁₋₆ alkylene group, (ii) —(CH₂)ₚ₁O—, (iii) —(CH₂)ₚ₁NH—, (iv) —(CH₂)ₚ₁S—, (v) —(CH₂)_{q1}CH(OH)(CH₂)_{q2}O—, (vi) —(CH₂)_{q1}CH(OH)(CH₂)_{q2}NH—, (vii) —(CH₂)_{q1}CH(OH)(CH₂)_{q2}S—, (viii) —(CH₂)ₚ₁CONH—, (ix) —COO(CH₂)ₚ₁O—, (x) —COO(CH₂)ₚ₁NH—, (xi) —COO(CH₂)ₚ₁S—, (xii) —(CH₂)_{q1}O(CH₂)_{q2}O—, (xiii) —(CH₂)_{q1}O(CH₂)_{q2}NH— or (xiv) —(CH₂)_{q1}O(CH₂)_{q2}S— (p¹is an integer of 1 to 6, and q¹ and q² are each an integer of 1 to 3), A is a nitrogen atom or CR^{7′} (R^{7′} is a hydrogen atom, a halogen atom, a C₁₋₆ alkyl group, a C₁₋₆ alkoxy-carbonyl group or a carboxyl group), R¹ is (1) a hydrogen atom, (2) a C₁₋₆ alkyl group optionally substituted by a group selected from the group consisting of (i) carboxyl, (ii) C₁₋₆ alkoxy-carbonyl, (iii) hydroxy and (iv) carbamoyl optionally having mono- or di-C₁₋₆ alkyl, (3) a C₆₋₁₄ aryl group, (4) a C₁₋₆ alkoxy group, (5) a C₁₋₆ alkoxy-carbonyl group, (6) a carboxyl group, (7) a carbamoyl group optionally having C₁₋₆ alkyl optionally substituted by carboxyl or C₁₋₆ alkoxy-carbonyl, or (8) a C₃₋₆ cycloalkyl group optionally substituted by C₁₋₆ alkoxy-carbonyl, R² is a hydrogen atom, a C₁₋₆ alkyl group, a C₁₋₆ alkoxy-carbonyl group or a carboxyl group, R³ is a hydrogen atom, R⁸ is a hydrogen atom or a hydroxy group is preferable.

Particularly, a compound wherein Ar¹ and Ar² are each a phenyl group, ring B is a ring represented by the formula

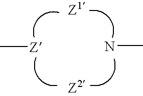

wherein Z′ is a methine group, and Z^{1′} and Z^{2′} are methylene group or ethylene group (preferably ethylene group), X is a bond, an oxygen atom or NH (preferably a bond or an oxygen atom), Y is a group represented by —(CH₂)ₚ₁NH— (p¹ is an integer of 1 to 6), A is CR^{7′} (R^{7′} is a hydrogen atom or a C₁₋₆ alkyl group), R¹ is (1) a hydrogen atom, (2) a C₁₋₆ alkyl group optionally substituted by carboxyl or C₁₋₆ alkoxy-carbonyl or (3) a carbamoyl group optionally having C₁₋₆ alkyl optionally substituted by C₁₋₆ alkoxy-carbonyl, R² is a hydrogen atom, R³ is a hydrogen atom, and R⁸ is a hydrogen atom, is preferable.

More specifically, (1) ethyl 2-[6-[3-[4-(diphenylmethoxy) piperidino]propylamino]imidazo[1,2-b]pyridazin-2-yl]-2-methylpropionate or a salt thereof (particularly, difumarate, disuccinate, citrate and the like), (2) 2-[6-[3-[4-(diphenylmethoxy)piperidino]propylamino]imidazo[1,2-b]pyridazin-2-yl]-2-methylpropionic acid or a salt thereof (particularly dihydrate), (3) ethyl N-[6-[3-[4-(diphenylmethoxy) piperidino]propylamino]imidazo[1,2-b]pyridazine-2-carbonyl]glycinate or a salt thereof, (4) ethyl 2-[6-[3-[4-(diphenylmethoxy) piperidino]propylamino]-3-methylimidazo[1,2-b]pyridazin-2-yl]-2-methylpropionate or a salt thereof (particularly dihydrochloride), (5) ethyl 2-[6-[3-[4-(diphenylmethylamino) piperidino]propylamino]imidazo[1,2-b]pyridazin-2-yl]-2-methylpropionate or a salt thereof, (6) 2-[6-[3-[4-(diphenylmethoxy)piperidino]propylamino]-3-methylimidazo[1,2-b]pyridazin-2-yl]-2-methylpropionic acid or a salt thereof, and (7) N-[6-[3-[4-(diphenylmethoxy)piperidino]propylamino]-3-methylimidazo[1,2-b]pyridazine-2-carbonyl]glycine or a salt thereof and the like are preferable.

And the compound represented by the formula:

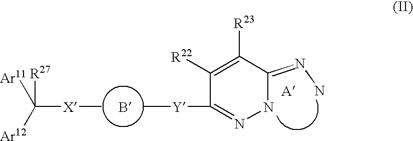

wherein ring A′ is a ring represented by the formula:

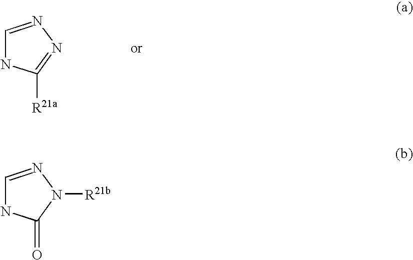

wherein R^{21a} is a hydrogen atom, a halogen atom, a hydrocarbon group optionally having a substituent, an acyl group or a hydroxy group having a substituent; R^{21b} is a hydrogen atom, a halogen atom, a hydrocarbon group optionally having a substituent, an acyl group or a hydroxy group optionally having a substituent; Ar¹¹ and Ar¹² are independently an aromatic group optionally having a substituent, and may form a condensed ring group with an adjacent carbon atom; ring B′ is a nitrogen-containing heterocycle optionally having a substituent; X′ and Y′, whether identical or not, are a bond, an oxygen atom, S(O)_{q} (q is an integer of 0 to 2), NR²⁴ wherein R²⁴ is a hydrogen atom or a lower alkyl group, or a divalent linear lower hydrocarbon group which may have a substituent, and which may contain 1 to 3 hetero atoms; R²² and R²³, whether identical or not, are a hydrogen atom, a halogen atom, a hydrocarbon group optionally having a substituent, an acyl group or a hydroxy group optionally having a substituent; R²⁷ is a hydrogen atom, a hydroxy group which may be substituted by lower alkyl or a carboxyl group; or a salt thereof exhibits excellent anti-allergic activity, anti-histaminic activity, anti-inflammatory activity, anti-PAF (platelet-activating factor) activity and eosinophil chemotaxis-inhibiting activity and the like. Therefore the compound can be used in the same way as compound (I) or its salt or its prodrug.

With respect to Formula (II) above, Compound (II) wherein ring A′ is Type (a) and Compound (II) wherein ring A′ is Type (b) are hereinafter referred to as Compound (IIa) and Compound (IIb), respectively.

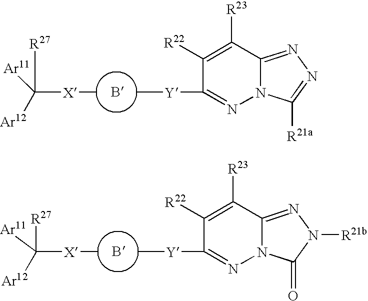

In Formula (II) above, Ar¹¹ and Ar¹² are an “aromatic group optionally having a substituent,” and may form a condensed ring group with an adjacent carbon atom.

Examples of the “aromatic group” represented by Ar¹¹ and Ar¹² include (1) monocyclic or condensed polycyclic aromatic hydrocarbon group, specifically 6- to 14-membered monocylic or condensed polycyclic aromatic hydrocarbon group such as C₆₋₁₄ aryl group (e.g., phenyl, tolyl, xylyl, biphenyl, 1-naphthyl, 2-naphthyl, 2-indenyl, 1-anthryl, 2-anthryl, 9-anthryl, 1-phenanthryl, 2-phenanthryl, 3-phenanthryl, 4-phenanthryl, 9-phenanthryl, etc.) (preferably phenyl, tolyl, xylyl, biphenyl, 1-naphthyl, 2-naphthyl, etc., particularly preferably phenyl, etc.), or (2) monocyclic group (preferably 5- to 8-membered) containing 1 or more (e.g., 1 to 4, preferably 1 to 3) of one or two kinds of hetero atoms selected from among a nitrogen atom, a sulfur atom and an oxygen atom, in addition to carbon atoms, or condensed aromatic heterocyclic group thereof, specifically aromatic heterocycle such as thiophene, benzo[b]thiophene, benzo[b]furan, benzimidazole, benzoxazole, benzothiazole, benzisothiazole, naphtho[2,3-b]thiophene, thianthrene, furan, isoindolylzine, oxanthrene, phenoxathiin, pyrrole, imidazole, triazole, thiazole, oxazole, pyrazole, pyridine, pyrazine, pyrimidine, pyridazine, indole, isoindole, 1H-indazole, purine, 4H-quinolizine, isoquinoline, quinoline, phthalazine, naphthylidine, quinoxaline, quinazoline, cinnoline, carbazole, β-carboline, phenarthridine, acridine, phenazine, isothiazole, phenothiazine, isoxazole, furaxan, phenoxazine and isochroman (preferably pyridine, thiophene, furan, etc., more preferably pyridine etc.), or monovalent group resulting from removal of an optionally selected hydrogen atom from a condensed ring formed by one of these rings (preferably monocyclic heterocycles mentioned above) and one or more than one (preferably 1 or 2, more preferably 1) aromatic ring (e.g., aromatic hydrocarbon groups mentioned above, preferably benzene ring, etc.).

The “aromatic group” of the “aromatic group optionally having a substituent” represented by Ar¹¹ and Ar¹² is preferably phenyl or the like.

Examples of the “substituent” for the aromatic group represented by Ar¹¹ and Ar¹² include: (i) halogen atom (e.g., fluorine, chlorine, bromine, iodine), (ii) lower alkylenedioxy group (e.g., C₁₋₃ alkylenedioxy group such as methylenedioxy and ethylenedioxy, and the like), (iii) nitro group, (iv) cyano group, (v) optionally halogenated lower alkyl group, (vi) optionally halogenated lower alkenyl group, (vii) optionally halogenated lower alkynyl group, (viii) lower cycloalkyl group (e.g., C₃₋₆ cycloalkyl group such as cyclopropyl, cyclobutyl, cyclopentyl and cyclohexyl, and the like), (ix) lower alkoxy group which may be substituted, (x) optionally halogenated lower alkylthio group, (xi) hydroxy group, (xii) amino group, (xiii) mono-lower alkylamino group (e.g., mono-C₁₋₆ alkylamino group such as methylamino, ethylamino, propylamino, isopropylamino and butylamino, and the like), (xiv) di-lower alkylamino group (e.g., di-C₁₋₆ alkylamino group such as dimethylamino, diethylamino, dipropylamino and dibutylamino, and the like), (xv) 5- or 6-membered cyclic amino group (e.g., morpholino, piperazin-1-yl, piperidino, pyrroridin-1-yl, and the like), (xvi) lower alkyl-carbonyl group (e.g., C₁₋₆ alkyl-carbonyl group such as acetyl, propionyl, and the like), (xvii) carboxyl group, (xviii) lower alkoxy-carbonyl group (e.g., C₁₋₆ alkoxy-carbonyl group such as methoxycarbonyl, ethoxycarbonyl, propoxycarbonyl, butoxycarbonyl,-and the like), (xix) carbamoyl group or thiocarbamoyl group, (xx) mono-lower alkyl-carbamoyl group (e.g., mono-C₁₋₆ alkyl-carbamoyl group such as methylcarbamoyl, ethylcarbamoyl, and the like) or mono-lower alkyl-thiocarbamoyl group (e.g., mono-C₁₋₆ alkyl-thiocarbamoyl group such as methylthiocarbamoyl, ethylthiocarbamoyl, and the like), (xxi) di-lower alkyl-carbamoyl group (e.g., di-C₁₋₆ alkylcarbamoyl group such as dimethylcarbamoyl, diethylcarbamoyl, and the like) or di-lower alkyl-thiocarbamoyl group (e.g., di-C₁₋₆ alkylthiocarbamoyl group such as dimethylthiocarbamoyl, diethylthiocarbamoyl, and the like), (xxii) aryl-carbamoyl (e.g., C₆₋₁₀ aryl-carbamoyl such as phenylcarbamoyl, naphthylcarbamoyl, and the like) or aryl-thiocarbamoyl (e.g., C₆₋₁₀ aryl-thiocarbamoyl such as phenylthiocarbamoyl, naphthylthiocarbamoyl, and the like), (xxiii) sulfo group, (xxiv) lower alkylsulfonyl group (e.g., C₁₋₆ alkylsulfonyl group such as methylsulfonyl, ethylsulfonyl, and the like), (xxv) aryl group (e.g., C₆₋₁₀ aryl group such as phenyl, naphthyl and the like), (xxvi) aryloxy group (e.g., C₆₋₁₀ aryloxy group such as phenyloxy, naphthyloxy, and the like), (xxvii) aralkyloxy group (e.g., C₇₋₁₆ aralkyloxy group such as benzyloxy and the like), (xxviii) alkyl-carbonyloxy group (e.g., C₁₋₆ alkyl-carbonyloxy group such as methylcarbonyloxy, ethylcarbonyloxy, propylcarbonyloxy, butylcarbonyloxy, isobutylcarbonyloxy, tert-butylcarbonyloxy, and the like) and (xxix) alkyl-carbonyloxy-alkoxy-carbonyl group (e.g., C₁₋₆ alkyl-carbonyloxy-C₁₋₆ alkoxy-carbonyl group such as methylcarbonyloxymethoxycarbonyl, methylcarbonyloxyethoxycarbonyl, ethylcarbonyloxymethoxycarbonyl, ethylcarbonyloxyethoxycarbonyl, and the like) and the like.

Examples of the “optionally halogenated lower alkyl group” include lower alkyl group (e.g., C₁₋₆ alkyl group such as methyl, ethyl, propyl, isopropyl, butyl, isobutyl, sec-butyl, tert-butyl, pentyl, hexyl and the like) optionally having 1 to 3 halogen atoms (e.g., fluorine, chlorine, bromine, iodine), specifically methyl, chloromethyl, difluoromethyl, trichloromethyl, trifluoromethyl, ethyl, 2-bromoethyl, 2,2,2-trifluoroethyl, propyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl, 6,6,6-trifluorohexyl, and the like.

Examples of the “optionally halogenated lower alkenyl group” and “optionally halogenated lower alkynyl group” include lower alkenyl group (e.g., C₂₋₆ alkenyl group such as vinyl, propenyl, isopropenyl, 2-buten-1-yl, 4-penten-1-yl, 5-hexen-1-yl, and the like) optionally having 1 to 3 halogen atoms (e.g., fluorine, chlorine, bromine, iodine) and lower alkynyl group (e.g., C₂₋₆ alkynyl group such as 2-butyn-1-yl, 4-pentyn-1-yl, 5-hexyn-1-yl, and the like) optionally having 1 to 3 halogen atoms (e. g., fluorine, chlorine, bromine, iodine).

Examples of the “lower alkoxy group which may be substituted” include lower alkoxy group (e.g., C₁₋₆ alkoxy group such as methoxy, ethoxy, propoxy, isopropoxy, n-butoxy, isobutoxy, sec-butoxy, tert-butoxy, and the like) optionally having 1 to 3 halogen atoms (e.g., fluorine, chlorine, bromine, iodine), mono- or di-lower alkylamino group (e.g., mono- or di-C₁₋₆ alkylamino group such as methylamino, dimethylamino, ethylamino and diethylamino) or lower alkoxy-carbonyl group (e.g., C₁₋₆ alkoxy-carbonyl group such as methoxycarbonyl, ethoxycarbonyl, and the like).

Examples of the “optionally halogenated lower alkylthio group” include lower alkylthio group (e.g., C₁₋₆ alkylthio group such as methylthio, ethylthio, n-propylthio, isopropylthio, n-butylthio, isobutylthio, sec-butylthio, tert-butylthio, and the like) optionally having 1 to 3 halogen atoms (e.g., fluorine, chlorine, bromine, iodine), specifically methylthio, difluoromethylthio, trifluoromethylthio, ethylthio, propylthio, isopropylthio, butylthio, 4,4,4-trifluorobutylthio, pentylthio, hexylthio, and the like.

Specific examples of the condensed ring formed by Ar¹¹ and Ar¹², along with the adjacent carbon atom, include condensed ring groups represented by the formula:

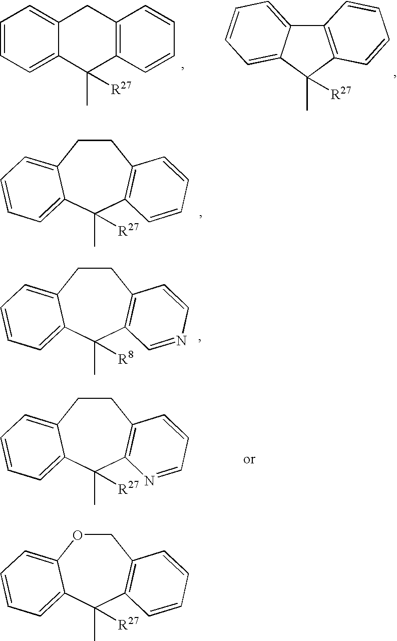

wherein R²⁷ has the same definition as that shown above.

It is preferable that Ar¹¹ and Ar¹², whether identical or not, are an aromatic hydrocarbon group (e.g., C₆₋₁₄ aromatic hydrocarbon group) optionally having a substituent, and a benzene ring optionally having a substituent is more preferred. More preferably, Ar¹¹ and Ar¹² are independently (1) phenyl group which may be substituted by a halogen atom or C₁₋₆ alkyl, or (2) a 5- to 8-membered aromatic heterocyclic group containing 1 to 4 hetero atoms selected from nitrogen atom, sulfur atom and oxygen atom, in addition to carbon atoms.

In Formula (II) above, ring B′ represents a “nitrogen-containing heterocycle optionally having a substituent”.

Examples of the “nitrogen-containing heterocycle” represented by ring B′ include 3- to 13-membered nitrogen-containing heterocycle which contains one nitrogen atom, which may further contain 1 to 3 hetero atoms selected from nitrogen atom, oxygen atom, sulfur atom, and the like. In Formula (II) above, it is preferable that ring B′ forms a divalent group resulting from removal of one hydrogen atom from the nitrogen atom and another atom of ring B′, respectively. Specific examples include 3- to 9-membered (more preferably 3- to 6-membered) nitrogen atom-containing heterocyclic groups such as

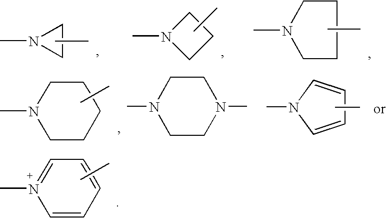

Examples of the substituent for the nitrogen-containing heterocycle represented by ring B′ include the same as the substituent for the “aromatic group optionally having a substituent” represented by Ar¹¹ and Ar¹² above and oxo group and the like.

Specific preferable examples of ring B′ include a ring represented by the formula:

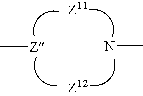

wherein Z″ is a nitrogen atom or a methine group, Z¹¹ and Z¹² are independently a linear C₁₋₄ alkylene group which may be substituted by a hydroxy group, an oxo group or a C₁₋₆ alkyl group.

Examples of said “C₁₋₆ alkyl group” include linear or branched C₁₋₆ alkyl group such as methyl, ethyl, propyl, isopropyl, butyl, isobutyl, sec-butyl, tert-butyl, pentyl, hexyl, and the like.

Examples of said “linear C₁₋₄ alkylene group” include linear C₁₋₄ alkylene group such as methylene, ethylene, propylene and butylene.

Preferable examples of the “linear C₁₋₄ alkylene group which may be substituted by a hydroxy group, an oxo group or a C₁₋₆ alkyl group” represented by Z¹¹ and Z¹² include unsubstituted linear C₁₋₄ alkylene group and the like, and unsubstituted linear C₁₋₂ alkylene groups are more preferred.

Ring B′ is more preferably piperidine, piperazine, and the like.

In Formula (II) above, X′ and Y′, whether identical or not, are (1) bond, (2) oxygen atom, (3) S(O)_{q} (q is an integer of 0 to 2), (4) NR²⁴ wherein R²⁴ is a hydrogen atom or a lower alkyl group, or (5) a divalent linear lower hydrocarbon group which may contain a substituent, and which may further contain 1 to 3 hetero atoms.

Examples of the lower alkyl group represented by R²⁴ include linear or branched C₁₋₆ alkyl group such as methyl, ethyl, propyl, isopropyl, butyl, isobutyl, sec-butyl, tert-butyl, pentyl, hexyl, and the like.

Examples of the “divalent linear lower hydrocarbon group which may further contain 1 to 3 hetero atoms” represented by X′ and Y′ include groups resulting from removal of each of hydrogen atoms (2 in total) bound to the same or different carbon atom from a lower (C₁₋₆) hydrocarbon, and which may optionally contain hetero atoms selected from oxygen atom, NR^{24′} wherein R^{24′} is a hydrogen atom or a lower alkyl group, sulfur atom, and the like, in the hydrocarbon chain.

Examples of the lower alkyl group represented by R^{24′} include linear or branched C₁₋₆ alkyl group such as methyl, ethyl, propyl, isopropyl, butyl, isobutyl, sec-butyl, tert-butyl, pentyl, hexyl, and the like.

Specific examples of the “divalent linear lower hydrocarbon group” include (i) C₁₋₆alkylene group (e.g., —CH₂—, —(CH₂)₂—, —(CH₂)₃—, —(CH₂)₄—, —(CH₂)₅—, —(CH₂)₆—, and the like), (ii) C₂₋₆ alkenylene group (e.g., —CH═CH—, —CH═CH—CH₂—, —CH₂—CH═CH—CH₂—, —(CH₂)₂—CH═CH—CH₂—, —(CH₂)₂—CH═CH—(CH₂)₂—, —(CH₂)₃—CH═CH—CH₂—, and the like) and (iii) C₂₋₆ alkynylene group (e.g., —C≡C—, —C≡C—CH₂—, —CH₂—C≡C—CH₂—, —(CH₂)₂—C≡C—CH₂—, —(CH₂)₂—C≡C—(CH₂)₂—, —(CH₂)₃—C≡C—CH₂—, and the like).

Examples of the “substituent” for the “divalent linear lower hydrocarbon group which may further contain 1 to 3 hetero atoms” represented by X′ and Y′ include the same as the “substituent” for the “aromatic group optionally having a substituent” represented by Ar¹¹ and Ar¹² above and oxo group and the like, and is preferably a hydroxy group or an oxo group.

X′ is preferably a bond, an oxygen atom or NH, and a bond or an oxygen atom is particularly preferred.

Preferable examples of Y′ include a group represented by the formula:

—(CH₂)ₛ—Y¹¹—(CH₂)ₜ—Y¹²—

wherein Y¹¹ and Y¹², whether identical or not, are a bond, an oxygen atom, S(O)_{q} wherein q has the same definition as that shown above, NR²⁴ wherein R²⁴ has the same definition as that shown above, a carbonyl group, a carbonyloxy group or a group represented by the formula:

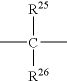

wherein R²⁵ and R²⁶, whether identical or not, are a hydroxy group or a C₁₋₄ alkyl group; s and t are independently an integer of 0 to 4 (sum of m and n is not more than 6).

Examples of the “C₁₋₄ alkyl group” represented by R²⁵ and R²⁶ include linear or branched C₁₋₄ alkyl group such as methyl, ethyl, propyl, isopropyl, butyl, isobutyl, sec-butyl, tert-butyl and the like.

Preferable examples of Y′ include (i) C₁₋₆ alkylene groups, (ii) —(CH₂)ₚ₂O—, (iii) —(CH₂)ₚ₂NH—, (iv) —(CH₂)ₚ₂S—, (v) —(CH₂)_{q3}CH(OH)(CH₂)_{q4}O—, (vi) —(CH₂)_{q3}CH(OH)(CH₂)_{q4}NH—, (vii) —(CH₂)_{q3}CH(OH)(CH₂)_{q4}S—, (viii) —(CH₂)ₚ₂CONH—, (ix) —COO(CH₂)ₚ₂O—, (x) —COO(CH₂)ₚ₂NH—, (xi) —COO(CH₂)ₚ₂S—, (xii) —(CH₂)_{q3}O(CH₂)_{q4}O—, (xii) —(CH₂)_{q3}O(CH₂)_{q4}NH— or (xiv) —(CH₂)_{q3}O(CH₂)_{q4}S— wherein p² is an integer of 1 to 6, q³ and q⁴ are an integer of 1 to 3.

In particular, Y′ is preferably a bond, —(CH₂)₂—O—, —(CH₂)₃—O—, —(CH₂)₄—O—, —(CH₂)₆—O—, —(CH₂)₂—NH—, —(CH₂)₃—NH—, —(CH₂)₄—NH—, —(CH₂)₃—S—, —CH₂—CH(OH)—CH₂—O—, —(CH₂)₂—CO—NH—, —CH₂—CO—NH—, —CO—O—(CH₂)₂—O—, —CO—O—(CH₂)₃—O—, —(CH₂)₆—NH—, —(CH₂)₆—S—, —(CH₂)₂—O—(CH₂)₂—O—, —(CH₂)₂—O—(—CH₂)₂—S—, and the like.

In the case of Compound (IIa), Y′ is preferably a group represented by the formula:

—(CH₂)ₛ—Y¹³—(CH₂)ₜ—Y¹⁴—

wherein Y¹³ is a bond or —CH(OH)—, Y¹⁴ is-an oxygen atom, S or NH, and s and t independently are an integer of 0 to 4 (sum of s and t is not more than 6). In particular, s and t are preferably an integer of 1 to 3, and 3 is more preferred. When Y¹³ is —CH(OH)—, s and t are preferably 1.

In the case of Compound (IIb), Y′ is preferably a group represented by the formula:

—(CH₂)_{w}—Y¹⁵—

wherein w is an integer of 1 to 6, and Y¹⁵ is an oxygen atom or NH. In particular, w is preferably an integer of 1 to 3, and 3 is more preferred.

In Formula (II) above, R^{21a} is a hydrogen atom, a halogen atom, a hydrocarbon group optionally having a substituent, an acyl group or a hydroxy group having a substituent.

R^{21b} is a hydrogen atom, a halogen atom, a hydrocarbon group optionally having a substituent, an acyl group or a hydroxy group optionally having a substituent.

R²² and R²³, whether identical or not, are a hydrogen atom, a halogen atom, a hydrocarbon group optionally having a substituent, an acyl group or a hydroxy group optionally having a substituent.

Examples of the “halogen atom” represented by R^{21a}, R^{21b}, R²² and R²³ include a fluorine atom, a chlorine atom, a bromine atom and an iodine atom.

Examples of the “hydrocarbon group” of the “hydrocarbon group optionally having a substituent” represented by R^{21a}, R^{21b}, R²² and R²³ include groups resulting from removal of one hydrogen atom from a hydrocarbon compound, specifically linear or cyclic hydrocarbon group such as alkyl group, alkenyl group, alkynyl group, cycloalkyl group, aryl group, aralkyl group, and the like. In particular, chain (linear or branched) or cyclic hydrocarbon groups, etc. having 1 to 16 carbon atoms are preferred, with greater preference given to

(a) alkyl group, preferably lower alkyl group (e.g., C₁₋₆ alkyl group such as methyl, ethyl, propyl, isopropyl, butyl, isobutyl, sec-butyl, tert-butyl, pentyl, hexyl, and the like),

(b) alkenyl group, preferably lower alkenyl group (e.g., C₂₋₆ alkenyl group such as vinyl, allyl, isopropenyl, butenyl, isobutenyl, sec-butenyl, and the like),

(c) alkynyl group, preferably lower alkynyl group (e.g., C₂₋₆ alkynyl group such as propargyl, ethynyl, butynyl, 1-hexynyl, and the like),

(d) cycloalkyl group, preferably lower cycloalkyl group (e.g., C₃₋₆ cycloalkyl group such as cyclopropyl, cyclobutyl, cyclopentyl, and cyclohexyl which may condense with a benzene ring optionally having 1 to 3 lower alkoxy groups (e.g., C₁₋₆ alkoxy groups such as methoxy and the like),

(e) aryl group (e.g., C₆₋₁₄ aryl group such as phenyl, tolyl, xylyl, biphenyl, 1-naphthyl, 2-naphthyl, 2-indenyl, 1-anthryl, 2-anthryl, 9-anthryl, 1-phenanthryl, 2-phenanthryl, 3-phenanthryl, 4-phenanthryl or 9-phenanthryl, preferably phenyl group), and

(f) aralkyl group (preferably lower aralkyl group (e.g., C₇₋₁₆ aralkyl group such as benzyl, phenethyl, diphenylmethyl, 1-naphthylmethyl, 2-naphthylmethyl, 2-phenylethyl, 2-diphenylethyl, 1-phenylpropyl, 2-phenylpropyl, 3-phenylpropyl, 4-phenylbutyl and 5-phenylpentyl and the like, more preferably benzyl group).

Examples of the “substituent” for said “hydrocarbon group” include the same as the “substituent” for the “aromatic group optionally having a substituent” represented by Ar¹¹ and Ar¹² above and oxo group and the like.

In particular, examples of preferred hydrocarbon include alkyl group such as C₁₋₆ alkyl group, and examples of substituents for hydrocarbon group include cyano, carboxyl, C₁₋₆ alkoxy-carbonyl, carbamoyl (or thiocarbamoyl), and the like.

Examples of the “acyl group” represented by R^{21a}, R^{21b}, R²² and R²³ include groups represented by the formula —(C═O)—R²⁸, —SO₂—R²⁸, —SO—R²⁸, —(C═O)NR²⁸R²⁹, —(C═O)O—R²⁸, —(C═S)O—R²⁸ or —(C═S)NR²⁸R²⁹ wherein R²⁸ is a hydrogen atom, a hydrocarbon group optionally having a substituent or a hydroxy group optionally having a substituent; and R²⁹ is a hydrogen atom or a lower alkyl group (e.g., C₁₋₆ alkyl group such as methyl, ethyl, propyl, isopropyl, butyl, isobutyl, sec-butyl, tert-butyl, pentyl, hexyl, and the like, preferably a C₁₋₃ alkyl group such as methyl, ethyl, propyl, isopropyl, and the like).

In particular, groups represented by the formula —(C═O)—R²⁸, —SO₂—R²⁸—, —SO—R²⁸, —(C═O)NR²⁸R²⁹ or —(C═O)O—R²⁸ are preferred, and a group represented by the formula —(C═O)—R²⁸ is more preferred.

The “hydrocarbon group which may be substituted” represented by R²⁸ is the same as the “hydrocarbon group optionally having a substituent” represented by R^{21a}, R^{21b}, R²² and R²³ above. In particular, the hydrocarbon group represented by R²⁸ is preferably an alkyl group such as a C₁₋₆ alkyl group, and the substituent thereof is preferably carboxyl, C₁₋₆ alkoxy-carbonyl, and the like. R²⁹ is preferably a hydrogen atom or the like.

Examples of the “hydroxy group having a substituent” represented by R^{21a} include hydroxy group having one group such as a hydrocarbon group optionally having a substituent, instead of a hydrogen atom of the hydroxy group.

Examples of the “hydroxy group optionally having a substituent represented by R^{21b}, R²², R²³ and R²⁸ include (1) a hydroxy group or (2) a hydroxy group having one group such as a hydrocarbon group optionally having a substituent, instead of a hydrogen atom of the hydroxy group.

The “hydrocarbon group optionally having a substituent” present in the hydroxy group is the same as the “hydrocarbon group optionally having a substituent” represented by R^{21a}, R^{21b}, R²², R²³ and R²⁸ above.

With respect to Compound (IIa), the acyl group represented by R^{21a}, R^{21b}, R²² and R²³ above is preferably (1) a carboxyl group, (2) a C₁₋₆ alkoxy-carbonyl group, (3) a carbamoyl group (or thiocarbamoyl group) which may be substituted by a C₁₋₆ alkyl group optionally having carboxyl or C₁₋₆ alkoxy-carbonyl, and the like.

In particular, R^{21a} is preferably (1) a hydrogen atom, (2) a carboxyl group, (3) a C₁₋₆ alkoxy-carbonyl group, (4) a C₁₋₆ alkyl group which may be substituted by a group selected from the group consisting of (i) cyano, (ii) carboxyl, (iii) C₁₋₆ alkoxy-carbonyl and (iv) carbamoyl (or thiocarbamoyl) or (5) a carbamoyl group (or thiocarbamoyl group) which may be substituted by a C₁₋₆ alkyl group optionally having carboxyl or C₁₋₆ alkoxy-carbonyl, or the like.

With respect to Compound (IIb), when R^{21b} is a hydrogen atom, the oxo group of the triazolo [4,3-b]pyridazine ring may be enolated, and the partial structural formula:

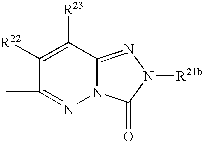

may represent any of the formula:

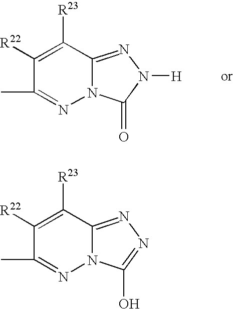

In particular, R^{21b} is preferably (1) a hydrogen atom, (2) a C₁₋₆ alkyl group which may be substituted by a group selected from the group consisting of (i) carboxyl, (ii) C₁₋₆ alkoxy-carbonyl, (iii) C₁₋₆ alkyl-carbonyloxy and (iv) C₁₋₆ alkyl-carbonyloxy-C₁₋₆ alkoxy-carbonyl, and the like.

With respect to Formula (II) above, R²²and R²³ are preferably a hydrogen atom.

In Formula (II) above, R²⁷ represents a hydrogen atom, a hydroxy group which may be substituted by a lower alkyl group or a carboxyl group.

Examples of the “lower alkyl group” of the “hydroxy group which may be substituted by a lower alkyl group” represented by R²⁷ include C₁₋₆alkyl group such as methyl, ethyl, propyl, isopropyl, butyl, isobutyl, sec-butyl, tert-butyl, pentyl, hexyl, and the like.

R²⁷ is preferably a hydrogen atom or a hydroxy group, and a hydrogen atom is particularly preferred.

As Compound (II) of the present invention, the following are preferred:

Compound (II)-I:

Compound (II) wherein R^{21a} is (1) a hydrogen atom, (2) a carboxyl group, (3) a C₁₋₆ alkoxy-carbonyl group, (4) a C₁₋₆ alkyl group which may be substituted by a group selected from the group consisting of (i) cyano, (ii) carboxyl, (iii) C₁₋₆ alkoxy-carbonyl and (iv) carbamoyl, or (5) a carbamoyl group which may be substituted by a C₁₋₆ alkyl group optionally having carboxyl or C₁₋₆ alkoxy-carbonyl; R^{21b} is (1) a hydrogen atom, or (2) a C₁₋₆ alkyl group which may be substituted by a group selected from the group consisting of (i) carboxyl, (ii) C₁₋₆ alkoxy-carbonyl, (iii) C₁₋₆ alkyl-carbonyloxy and (iv) C₁₋₆ alkyl-carbonyloxy-C₁₋₆ alkoxy-carbonyl; R²² and R²³ are a hydrogen atom; R²⁷ is a hydrogen atom or a hydroxy group (particularly a hydrogen atom); Ar¹¹ and Ar¹²are independently a phenyl group which may be substituted; ring B′ is a ring represented by the formula:

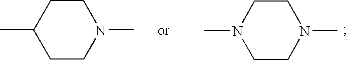

X′ is a bond or an oxygen atom; Y′ is a group represented by the formula:

—(CH₂)ₛ—Y¹³—(CH₂)ₜ—Y¹⁴—

wherein Y¹³ is a bond or —CH(OH)—, Y¹⁴ is an oxygen atom, S or NH, and s and t are independently an integer of 0 to 6 (sum of s and t is not more than 6).

Compound (II)-II:

(1) 6-[6-[4-(diphenylmethoxy)piperidino]hexyloxy][1,2,4]triazolo[4,3-b]pyridazine or a salt thereof, (2) 6-[6-[4-(diphenylmethoxy)piperidino]hexylamino][1,2,4]triazolo[4,3-b]pyridazine or a salt thereof, (3) 3-tert-butyl-6-[3-[4-(diphenylmethoxy)piperidino]propoxy][1,2,4]triazolo[4,3-b]pyri dazine or a salt thereof, (4) 6-[3-[4-(diphenylmethoxy) piperidino]propylamino][1,2,4]triazolo[4,3-b]pyridazine-3-carboxylic acid or a salt thereof, (5) 6-[3-[4-(diphenylmethoxy) piperidino]propylamino][1,2,4]triazolo[4,3-b]pyridazin-3(2H)-o ne or a salt thereof, (6) Ethyl 2-[6-[3-[4-(diphenylmethoxy) piperidino]propylamino]-3-oxo-[1,2,4]triazolo[4,3-b]pyridazin-2(3H)-yl]-2-methylpropionate or a salt thereof, (7) 2-[6-[3-[4-(diphenylmethoxy)piperidino]propylamino]-3-oxo-[1,2,4]triazolo [4,3-b]pyridazin-2(3H)-yl]-2-methylpropionic acid or a salt thereof, (8) Pivaloyloxymethyl 2-[6-[3-[4-(diphenylmethoxy) piperidino]propylamino]-3-oxo-[1,2,4]triazolo[4,3-b]pyridazin-2(3H)-yl]-2-methylpropionate or a salt thereof, (9) Pivaloyloxymethyl 2-[6-[3-[4-(diphenylmethoxy)piperidino]propoxy]-3-oxo-[1,2,4]triazolo[4,3-b]pyridazin-2(3H)-yl]-2-met hylpropionate or a salt thereof.

As Compound (IIa) of the present invention, the following are preferred:

Compound (IIa)-I:

Compound (IIa) wherein Ar¹¹ and Ar¹² are independently a phenyl group which may be substituted; ring B′ is a ring represented by the formula:

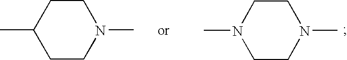

X′ is a bond or an oxygen atom; Y′ is a group represented by the formula:

—(CH₂)ₛ—Y¹³—(CH₂)ₜ—Y¹⁴—

wherein Y¹³ is a bond or —CH(OH)—, Y¹⁴ is an oxygen atom, S or NH, and s and t are independently an integer of 0 to 4 (sum of s and t is not more than 6);

R^{21a} is (1) a hydrogen atom, (2) a carboxyl group, (3) a C₁₋₆ alkoxy-carbonyl group, (4) a C₁₋₆ alkyl group which may be substituted by a group selected from the group consisting of (i) cyano, (ii) carboxyl, (iii) C₁₋₆ alkoxy-carbonyl and (iv) carbamoyl (or thiocarbamoyl), or (5) a carbamoyl group (or thiocarbamoyl group) which may be substituted by a C₁₋₆ alkyl group optionally having carboxyl or C₁₋₆ alkoxy-carbonyl; and R²², R²³ and R²⁷ are a hydrogen atom.

Compound (IIa)-II:

(1) 6-[6-[4-(diphenylmethoxy)piperidino]hexyloxy][1,2,4]triazolo[4,3-b]pyridazine or a salt thereof, (2) 6-[6-[4-(diphenylmethoxy)piperidino]hexylamino][1,2,4]triazolo [4,3-b]pyridazine or a salt thereof, (3) 3-tert-butyl-6-[3-[4-(diphenylmethoxy)piperidino]propoxy][1,2,4]triazolo[4,3-b]pyridazine or a salt thereof, (4) 6-[3-[4-(diphenylmethoxy) piperidino]propylamino][1,2,4]triazolo[4,3-b]pyridazine-3-carboxylic acid or a salt thereof.

As Compound (IIb) of the present invention, the following are preferred:

Compound (IIb)-I:

Compound (IIb) wherein Ar¹¹ and Ar¹² are independently a phenyl group which may be substituted; ring B′ is a ring represented by the formula:

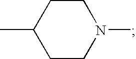

X′ is an oxygen atom; Y′ is a group represented by the formula:

—(CH₂)_{w}—Y¹⁵—

wherein w is an integer of 1 to 6, and Y¹⁵ is an oxygen atom or NH; R^{21b} is (1) a hydrogen atom, or (2) a C₁₋₆ alkyl group which may be substituted by a group selected from the group consisting of (i) carboxyl, (ii) C₁₋₆ alkoxy-carbonyl, (iii) C₁₋₆ alkyl-carbonyloxy and (iv) C₁₋₆ alkyl-carbonyloxy-C₁₋₆ alkoxy-carbonyl; and R²², R²³ and R²⁷ are a hydrogen atom.

Compound (Ib)-II:

(1) 6-[3-[4-(diphenylmethoxy)piperidino]propylamino][1,2,4]triazolo[4,3-b]pyridazin-3(2H)-one or a salt thereof, (2) Ethyl 2-[6-[3-[4-(diphenylmethoxy)piperidino]propylamino]-3-oxo-[1,2 ,4]triazolo[4,3-b]pyridazin-2(3H)- yl]-2-methylpropionate or a salt thereof, (3) 2-[6-[3-[4-(diphenylmethoxy)piperidino]propylamino]-3-oxo-[1,2,4]triazolo[4,3-b]pyridazin-2(3H)-yl]-2-methylpropionic acid or a salt thereof, (4) Pivaloyloxymethyl 2-[6-[3-[4-(diphenylmethoxy)piperidino]propylamino]-3-oxo-[1,2 ,4]triazolo[4,3-b]pyridazin-2(3H)-yl]-2-methylpropionate or a salt thereof, (5) Pivaloyloxymethyl 2-[6-[3-[4-(diphenylmethoxy) piperidino]propoxy]-3-oxo-[1,2,4]triazolo[4,3-b]pyridazin-2(3H )-yl]-2-methylpropionate or a salt thereof.

The compound (I) or salt thereof can be produced in accordance with the known methods. For example, the compound (I) or salt thereof can be produced by the method described in JP-A-2000-191663, JP-A-2000-191664, JP-A-2000-198735 and WO 00/23450 or the applied methods thereof.

The compound (II) or salt thereof can be produced in accordance with the known methods. For example, the compound (II) or salt thereof can be produced by the method described in JP-A-2000-178277 and WO 00/20417 or the applied methods thereof.

Salts of the compound (I) or (II) include, for example, salt with inorganic acid (e.g., hydrochloric acid, phosphoric acid, hydrobromic acid, sulfuric acid, and the like) and salt with organic acid (e.g., acetic acid, formic acid, propionic acid, fumaric acid, maleic acid, succinic acid, tartaric acid, citric acid, malic acid, oxalic acid, methanesulfonic acid, benzenesulfonic acid, and the like). Provided that the compound (I) or (II) has an acidic group such as carboxylic acid and the like as a substituent thereof, the acidic group may form a salt with an inorganic base (e.g., an alkali metal or alkaline earth metal such as sodium, potassium, calcium, magnesium, and the like, or ammonia) or an organic base (e.g., tri-C₁₋₃ alkylamine such as triethylamine, and the like).

As the “acidic compound” used in the present invention, either of an acidic compound which is solid at a normal temperature (15 to 25° C.) or an acidic compound which is liquid at a normal temperature may be used, and it is preferable to use an acidic compound which is solid at a normal temperature. For enhancing the chemical stability and the dissolution property, the acidic compound is preferably in the form of a particle in which 50% or more of constituting particles are particles of 50 μm to 1.5 mm. Inter alia, the acidic compound in the form of a particle in which constituting particles are particles of 150 μm to 1.0 mm is preferable. Since the present acidic compound exerts the better effect as the content of a fine particle becomes smaller, a preferable example of the acidic compound is an acidic compound in the form of a particle in which particles of 50 μm or smaller among constituting particles are 20% or less of the all particles.

In addition, examples of the acidic compound include carboxylic acid, sulfonic acid, acidic polysaccharide and acidic amino acid, and the acidic compound may be hydrated or anhydrous. Examples thereof include carboxylic acids such as acetic acid, lactic acid, fumaric acid, tartaric acid, succinic acid, citric acid (in particular, citric acid (anhydrous)), oxalic acid, malonic acid, maleic acid, dl-malic acid, stearic acid, adipic acid and the like, sulfonic acids such as aminoethylsulfonic acid and the like, acidic polysaccharides such as aliginic acid and the like, acidic amino acids such as gultamic acid, aspartic acid and the like, and salts of amino acid and mineral acid such as glycine hydrochloride, aspartic acid hydrochloride and gultamic acid hydrochloride, and these may be used alone or in combination of 2 or more.

Among the acidic compounds, carboxylic acid is preferable, and fumaric acid, adipic acid, malic acid, acetic acid, tartaric acid, succinic acid and citric acid are preferable. Inter alia, tartaric acid, succinic acid and citric acid which are carboxylic acid being solid at a normal temperature (15 to 25° C.) are preferable and, in particular, citric acid is preferable.

The acid-blended preparation of the present invention contains a compound having the anti-allergy activity, the anti-histamine activity, the anti-inflammatory activity, the anti-PAF activity and/or the eosinophile chemotaxis inhibiting activity, and an acidic compound.

An amount of the acidic compound to be used in the acid-blended preparation of the present invention is about 0.01 to 100 parts by weight, preferably about 0.1 to 10 parts by weight, more preferably 0.5 to 2 parts by weight relative to 1 part by weight of the compound having the anti-allergy activity, the anti-histamine activity, the anti-inflammatory activity, the anti-PAF activity and/or the eosinophile chemotaxis inhibiting activity. The acidic compound in the acid-blended preparation of the present invention is incorporated for the purpose of decreasing a local pH at a part at which the preparation is present in stomach, and for the purpose of solubilizing a physiologically active substance. Therefore, the acidic compound can be used at a smaller amount than that normally used.

The acid-blended preparation of the present invention may further contain an excipient, a disintegrant, a binder, a lubricant, a colorant, a flavor and a light-shielding agent which are the conventional preparation material.

Examples of the “excipient” include lactose, starch, sucrose, mannit, crystalline cellulose, colloidal anhydrous silica, magnesium carbonate, calcium carbonate, calcium phosphate, calcium sulfate, aluminium silicate and aluminium metasilicate, and the like.

Examples of the “disintegrant” include calcium carboxymethylcellulose, croscarmellose sodium, sodium carboxymethylstarch, starch, low substituted hydroxypropylcellulose and cross-linked insoluble polyvinylpyrrolidone, and the like.

Examples of the “binder” include hydroxypropylcellulose, pregelatinized starch, sucrose, gelatin, gum arabic powder, methylcellulose, hydroxypropylmethylcellulose, carboxymethylcellulose, sodium carboxymethylcellulose, polyvinylpyrrolidone, crystalline cellulose, dextrin and pullulan, and the like.

Examples of the “lubricant” include stearic acid, calcium stearate, magnesium stearate, talc and colloidal silica, and the like.

Examples of the “colorant” include yellow iron sesquioxide and red ferric oxide, and the like.

The “flavor” may be synthetic or natural flavor and include lemon flavor, lime flavor, orange flavor, strawberry flavor and menthol, and the like.

The examples of the “light-shielding agent” include titanium oxide, talc, calcium carbonate and magnesium carbonate, and the like.

The content of the excipient in the acid-blended preparation of the present invention is not particularly limited as far as the object of the present invention is accomplished, but is, for example, about 1 to 99.9% by weight, preferably about 20 to 90% by weight.

The content of the disintegrant in the acid-blended preparation of the present invention is not particularly limited as far as the object of the present invention is accomplished, but is, for example, about 0.05 to 50% by weight, preferably about 0.2 to 20% by weight.

The content of the binder in the acid-blended preparation of the present invention is not particularly limited as far as the object of the present invention is accomplished, but is, for example, about 0.05 to 50% by weight, preferably about 0.2 to 20% by weight.

The content of the lubricant in the acid-blended preparation of the present invention is not particularly limited as far as the object of the present invention is accomplished, but is, for example, about 0.1 to 10% by weight, preferably about 0.3 to 3% by weight.

The content of the colorant in the acid-blended preparation of the present invention is not particularly limited as far as the object of the present invention-is-accomplished, but is, for example, about 0.001 to 10% by weight, preferably about 0.001 to 1% by weight.

The content of the flavor in the acid-blended preparation of the present invention is not particularly limited as far as the object of the present invention is accomplished, but is, for example, about 0.001 to 10% by weight, preferably about 0.001 to 1% by weight.

The content of the light-shielding agent in the acid-blended preparation of the present invention is not particularly limited as far as the object of the present invention is accomplished, but is, for example, about 0.02 to 20% by weight, preferably about 0.05 to 5% by weight.

Preferable embodiments of the acid-blended preparation of the present invention include:

(1) an acid-blended preparation comprising a compound (I) or (II) or a salt thereof, and tartaric acid,

(2) an acid-blended preparation comprising a compound (I) or (II) or a salt thereof, tartaric acid, lactose and corn starch,

(3) an acid-blended preparation comprising a compound (I) or (II) or a salt thereof, tartaric acid, lactose, corn starch, hydroxypropylcellulose and crystalline cellulose,

(4) an acid-blended preparation comprising a compound (I) or (II) or a salt thereof, tartaric acid, lactose, corn starch, hydroxypropylcellulose, crystalline cellulose, croscarmellose sodium and magnesium stearate,

(5) an acid-blended preparation comprising a compound (I) or (II) or a salt thereof, and succinic acid,

(6) an acid-blended preparation comprising a compound (I) or (II) or a salt thereof, succinic acid, lactose and corn starch,

(7) an acid-blended preparation comprising a compound (I) or (II) or a salt thereof, succinic acid, lactose, corn starch, hydroxypropylcellulose and crystalline cellulose,

(8) an acid-blended preparation comprising a compound (I) or (II) or a salt thereof, succinic acid, lactose, corn starch, hydroxypropylcellulose, crystalline cellulose, croscarmellose sodium and magnesium stearate,

(9) an acid-blended preparation comprising a compound (I) or (II) or a salt thereof, and citric acid (anhydrous),

(10) an acid-blended preparation comprising a compound (I) or (II) or a salt thereof, citric acid (anhydrous), lactose and corn starch,

(11) an acid-blended preparation comprising a compound (I) or (II) or a salt thereof, citric acid (anhydrous), lactose, corn starch, hydroxypropylcellulose and crystalline cellulose,

(12) an acid-blended preparation comprising a compound (I) or (II) or a salt thereof, citric acid (anhydrous), lactose, corn starch, hydroxypropylcellulose, crystalline cellulose, croscarmellose sodium and magnesium stearate; and the like.

In addition, preparations in which talc is contained in these acid-blended preparations are also preferable examples.

The acid-blended preparation of the present invention can be prepared, for example, by the method described below.

A compound having the anti-allergy activity, the anti-histamine activity, the anti-inflammatory activity, the anti-PAF activity and/or the eosinophile chemotaxis inhibiting activity (hereinafter, abbreviated as physiologically active substance in some cases) is mixed with a binder and granulated to prepare a granule (physiologically active substance granule). Similarly, an acidic compound and a binder are mixed and granulated to prepare a granule (acidic compound granule). Mixing and granulation are carried out using a normally used granulated machine. Upon this, a mixture (premix) obtained by mixing an excipient and/or a disintegrant and a physiologically active substance or an acidic substance in advance, and a binder may be mixed and granulated. Mixing of a physiologically active substance or an acidic compound and a binder, and granulation are carried out, preferably, at about 0 to 100° C. The content of the binder to be used in a granule is about 0.1 to 50% by weight. The contents of the excipient and the disintegrant to be used in a granule are about 1 to 99.9% by weight and about 0.1 to 50% by weight, respectively. The resulting granule contains particles of 50 μm to 1.5 mm at an amount of 50% or more (preferably, particles of 150 μm to 1.0 mm at an amount of 50% or more). For the purpose of removing a moisture, the resulting granule may be dried at about 10 to 80° C. far about 0.01 to 72 hours. Further, a particle size of the prepared granule may be adjusted. In particle size adjustment, a commercially available particle size adjusting machine such as a power mill is usually used. A granule after particle size adjustment contains particles of about 50 μm to 1.5 mm at an amount of 50% or more (preferably particles of 150 μm to 1.0 mm at an amount of 50% or more).

The physiologically active substance granule and the acidic compound granule which have been thus prepared may be usually used by mixing (mixed granule) or may be used separately. When used by mixing, it is preferable that the physiologically active substance granule and the acidic compound granule contain particles of 10 μm to 2.0 mm at an amount of 50% or more. More preferably, those granules contain particles of 50 μm to 1.5 mm at an amount of 50% or more, particularly preferably those granules contain particles of 150 μm to 1.0 mm at an amount of 50% or more. A disintegrant such as croscarmellose sodium and a lubricant such as magnesium stearate may be further added thereto. In addition, an acidic compound powder may be used as it is without using the acidic compound granule. In mixing of them, a commercially available mixing machine such as a tumbler mixing machine is usually used. An amount of a disintegrant to be used and the content of a lubricant are slightly larger than amounts which are used for normal preparations, and are about 0.1 to 50% by weight and about 0.1 to 10% by weight, respectively.

Although the resulting mixed granule may be used as it is as a granule, it is usually tailored into a dosage form such as a pill, a tablet and a capsule. The mixed granule is molded into a tablet such as a circular tablet and an oval type tablet, more preferably an oval type tablet. In molding, a commercially available molding machine such as a tabletting machine is used. A tabletting pressure upon molding into a tablet is usually about 1 to 25 kN. A circular tablet has usually a diameter of about 5 to 20 mm and a thickness of about 1 to 10 mm. An oval type tablet has usually a major axis of about 7 to 20 mm, a minor axis of about 5 to 15 mm, and a thickness of about 1 to 10 mm. In order to formulate the resulting tablet into a coated preparation, the tablet may be further subjected to film coating. For film coating procedures, a pan coating machine is usually used. Examples of a film-coated tablet include a film-coated circular tablet and a film-coated oval type tablet, preferably a film-coated oval type tablet.

A film coating solution can be prepared by dissolving or suspending a polymer for film coating such as hydroxypropylmethylcellulose in a solvent such as water. It is preferable that a colorant and a light-shielding agent are further incorporated into the film coating solution. It is preferable that a temperature of a product (tablet) at film coating solution spraying is controlled at about 10 to 100° C. It is more preferable to control at about 30 to 80° C., and it is more preferable to control at about 40 to 60° C.

In addition, examples of the case where a physiologically active substance granule and an acidic compound granule are separately used without mixing include a separately packed granule in which a physiologically active substance granule and an acidic compound granule are separately packed and a multi-layered tablet or a bilayered tablet comprising a layer of a physiologically active substance granule and a layer of an acidic compound granule, and the like.

Further, upon formulation of the physiologically active substance in the present invention into a preparation, a solid dispersion preparation and an oily preparation are also an example of a preparation.

The acid-blended preparation of this invention is superior in absorbability, stability and also dissolution property of the physiologically active substance.

The acid-blended preparation of this invention can be safely used as a drug in mammals (e.g., human, mice, dogs, rats, bovins, and the like), because it exhibits low toxicity.

The acid-blended preparation of this invention contains a compound having anti-allergy activity, anti-histaminic activity, anti-inflammatory activity, anti-PAF (platelet-aggregating factor) activity, eosinophil chemotaxis-inhibiting activity and the like, therefore, it can be used for prophylaxis and treatment of allergic dermatitis such as eczama, dermatitis, contact dermatitis, itching, dry dermatitis, acute urticaria, prurigo and the like; inflammatory dermatitis such as atopic dermatitis and the like; and the like. Furthermore, the acid-blended preparation of this invention can be used for prophylaxis and treatment of increase of intranasal pressure, sneeze, nasal secretion, pollinosis, hypersensitivity of uper respiratory tract and the like, and also for improvement of snuff.

The dosage of the acid-blended preparation of this invention differs in accordance with the kind and the amount of the physiologically active substance, the dosage form, the duration time of the phisiologically active substance emission, the objective disease, the objective animal, and the like. For example, when compound (I) or salt thereof is used as a physiologically active substance, the daily dose of the effective ingredient (compound (I) or salt thereof) is generally about 0.1 to about 100 mg/kg, preferably about 1 to about 50 mg/kg, more prefereably about 1 to 10 mg/kg for an adult patient (60 kg weight) once or twice a day.

The present invention is explained in detail in the folowing by referring to Reference Examples, Examples and Assesment Examples, which are not to be construed as limitative.

“Cornstarch”, “hydroxypropylcellulose (HPC-L)”, “magnesium stearate”, “tartaric acid”, “citric acid (anhydride)”, “microcrystalline cellulose”, “colloidal anhydrous silica”, “polyvinylpyrrolidone”, “lactose”, “sterile talc”, “croscarmellose sodium (Ac-Di-Sol)”, “hydroxypropylmethylcellulose”, “polyethyleneglycol 6000”, “red ferric oxide” and “titanium oxide” used in the Examples are the compounds conformed to 14^{th} revision Japanese Pharmacopoeia. And “succinic acid” used in the Examples is the high quality reagent.

### EXAMPLES

### Reference Example 1

### Production of ethyl 2-[6-[3-[4-(diphenylmethoxy)piperidino]propylamino]imidazo[1,2-b]pyridazin-2-yl]-2-methylpropionate difumarate

4.2 g of 4-(diphenylmethoxy)-1-piperidinepropaneamine and 1.76 g of ethyl 2-(6-chloroimidazo[1,2-b]pyridazin-2-yl)-2-methylpropionate were stirred at 192-200° C. for 3.5 hours. After cooling, an aqueous sodium bicarbonate solution was added thereto and extracted with ethyl acetate. The extract was washed with a brine solution, dried over magnesium sulfate and concentrated under reduced pressure. The residue was subjected to silica gel column chromatography to be eluted with ethyl acetate: methanol:triethyl amine (100:5:1). The desired fraction was collected and dissolved in 16 mL of ethyl acetate, and a solution prepared by dissolving 867 mg of fumaric acid in 16 mL of methanol was added thereto, followed by concentration. To the residue was added acetone and the crystals formed were collected by filtration, washed with acetone and dried to give 2.30 g of the title compound. Melting point 126-128° C.

Elemental analysis: for C₄₁H₄₉N₅O₁₁. Calculated (%): C, 62.50 ; H, 6.27 ; N, 8.89. Found (%): C, 62.28 ; H, 6.15; N. 8.97.

### Reference Example 2

### Production of ethyl 2-[6-[3-[4-(diphenylmethoxy)piperidino]propylamino]imidazo[1,2-b]pyridazin-2-yl]-2-methylpropionate disuccinate

In 1 mL of ethanol, 0.278 g of the ethyl 2-[6-[3-[4-(diphenylmethoxy)piperidino]propylamino]imidazo[1,2-b]pyridazin-2-yl]-2-methylpropionate produced in Reference Example 1 was dissolved, and 0.118 g of succinic acid was added thereto and dissolved, followed by concentration under reduced pressure. To the residue was added 0.5 mL of tetrahydrofuran and the residue was dissolved. After the addition of 2 mL of ethyl acetate, crystals formed were collected by filtration, washed with ethyl acetate and dried to give 0.382 g of the title compound.

Melting point 98-101° C. (decomposed) Elemental analysis: for C₄₁H₅₃N₅O₁₁.⅓CH₃CO₂C₂H₅. Calculated (%): C, 61.92 ; H, 6.83 ; N, 8.53. Found (%): C, 61.54 ; H, 6.83 ; N, 8.50.

### Reference Example 3

### Production of ethyl 2-[6-[3-[4-(diphenylmethoxy)piperidino]propylamino]imidazo[1,2-b]pyridazin-2-yl]-2-methylpropionate citrate (1:1)

In 8 mL of ethanol, 1.667 g of the ethyl 2-[6-[3-[4-(diphenylmethoxy)piperidino]propylamino]imidazo[1,2-b]pyridazin-2-yl]-2-methylpropionate produced in Reference Example 1 was dissolved, and 0.631 g of citric acid monohydrate was added thereto and dissolved under heating, followed by concentration under reduced pressure. To the residue was added 23 mL of ethyl acetate, and the crystals formed were collected by filtration and washed with 12 mL of ethyl acetate. To the crystals was added 30 mL of methanol and the crystals were dissolved under heating, followed by concentration under reduced pressure. To the residue was added 30 mL of ethanol and the residue was then dissolved. After still standing, the crystals formed were collected by filtration, washed with 10 mL of ethanol and dried to give 2.01 g of the title compound.

Melting point 176° C. (decomposed) Elemental analysis: for C₃₉H₄₉N₅O₁₀. Calculated (%): C, 62.64 ; H, 6.60 ; N, 9.36. Found (%): C, 62.50; H, 6.56, N, 9.43.

### Reference Example 4

### Production of 2-[6-[3-[4-(diphenylmethoxy)piperidino]propylamino]imidazo[1,2-b]pyridazin-2-yl]-2-methylpropionic acid

468 mg of ethyl 2-[6-[3-[4-(diphenylmethoxy)piperidino]propylamino]imidazo[1,2-b]pyridazin-2-yl]-2-methylpropionate was dissolved in 3 ml of ethanol; 2 ml of 1 N aqueous solution of sodium hydroxide was added, followed by stirring at room temperature for 15 hours. After the mixture was concentrated under reduced pressure, the residue was diluted with water and washed with ethyl acetate; the water layer was adjusted to pH 7 by the addition of 1 N hydrochloric acid, followed by extraction with ethyl acetate-tetrahydrofuran (1:1); the extract was washed with saturated brine, dried over magnesium sulfate and concentrated under reduced pressure. Ethyl acetate was added to the residue; the crystals precipitated were collected by filtration, washed with ethyl acetate and dried to give 267 mg of the title compound. The crystals can be recrystalized from acetone.

Melting point : 205-206° C. Elemental analysis: for C₃₁H₃₇N₅O₃. Calculated (%): C, 70.56; H, 7.07; N, 13.27. Found (%) : C, 70.46; H, 7.06; N, 13.36.

### Reference Example 5

### Production of sodium 2-[6-[3-[4-(diphenylmethoxy)piperidino]propylamino]imidazo[1,2-b]pyridazin-2-yl]-2-methylpropionate

To a solution of 2-[6-[3-[4-(diphenylmethoxy)piperidino]propylamino]imidazo[1,2-b]pyridazin-2-yl]-2-methylpropionic acid (528 mg) in methanol (2 ml), 2 N aqueous solution of sodium hydroxide (0.47. ml) was added, followed by stirring at room temperature for 5 minutes. This solution was diluted with 2-propanol and concentrated under reduced pressure. The residue was dissolved in 2-propanol and again concentrated under reduced pressure. To this residue, 2-propanol and ethyl ether were added; the resulting powder was collected by filtration to give the title compound (474 mg).

Amorphous

Elemental analysis: for C₃₁H₃₆N₅O₃Na.0.5H₂O. Calculated (%): C, 66.65; H, 6.68; N, 12.54. Found (%) : C, 66.45; H, 6.54; N, 12.53.

### Reference Example 6

### Production of 2-[6-[3-[4-(diphenylmethoxy)piperidino]propylamino]imidazo[1,2-b]pyridazin-2-yl]-2-methylpropionic acid dihydrate (hereinafter referred to briefly as compound A)

In 600 mL of dimethyl sulfoxide were suspended 363.6 g (1120 mmol) of 4-(diphenylmethoxy)-1-piperidinepropaneamine, 200.0 g (747 mmol) of ethyl 2-(6-chloroimidazo[1,2-b]pyridazin-2-yl)-2-methylpropionate and 158.4 g (1490 mmol) of sodium carbonate, which were then heated in an oil bath (bath temperature 165-170° C.) under a nitrogen gas stream and stirred for 3.5 hours. After cooling to room temperature, 2000 mL of ethyl acetate and 2000 mL of water were added, followed by separation into two layers. The organic layer was washed with 1000 mL of water twice and concentrated under reduced pressure. To the residue was added 1000 mL of ethanol and concentrated under reduced pressure to give 588 g of crude ethyl 2-[6-[3-[4-(diphenylmethoxy)piperidino]propylamino]imidazo[1,2-b]pyridazin-2-yl]-2-methylpropionate as an oil. This oil was dissolved in 1400 mL of ethanol, and 59.8 g (1490 mmol) of sodium hydroxide dissolved in 600 mL of water was added thereto. The reaction mixture was heated to 60° C. (inner temperature) and stirred for 1 hour. The reaction solution was concentrated under reduced pressure. To the residue were added 2000 mL of water and 2000 mL of ethyl acetate, followed by separation into two layers. The aqueous layer was washed with 1000 mL of ethyl acetate twice, and 2000 mL of ethanol was added to the aqueous layer. After the aqueous layer was adjusted to about pH 6 by the addition of 1000 mL of 1N hydrochloric acid, the crystals formed were collected by filtration, washed with 800 mL of water and 800 mL of ethanol:water (1:1), and dried to give 353.6 g of the crude title compound. HPLC purity area percentage 97.7%, Yield 82.0%

To 353.6 g of the crude title compound thus obtained was added 1240 mL of ethanol, and heated under reflux for 1 hour. The solution was stirred under ice-cooling. The crystals formed were collected by filtration, washed with 930 mL of cold ethanol and dried. The resulting crystals were suspended in 2000 mL of water and stirred for 1 hour while heating in a water bath (inner temperature 65-70° C.). After cooling to room temperature, the crystals formed were collected by filtration, washed with 1000 mL of water and dried to give 276 g of the title compound.

Melting point 203-205° C. (The crystals began to soften at 110-120° C. and solidified again.). Elemental analysis: for C₃₁H₃₇N₅O₃.2H₂O. Calculated (%): C, 66.05 ; H, 7.33 ; N, 12.42. Found (%): C, 66.35 ; H, 7.29 ; N, 12.39.

### Reference Example 7

### Production of N-[6-[3-[4-(diphenylmethoxy)piperidino]propylamino]imidazo[1,2-b]pyridazine-2-carbonyl]glycine ethyl ester

1.90 g of 4-(diphenylmethoxy)-1-piperidinepropanamine and 1.38 g of N-(6-chloroimidazo[1,2-b]pyridazine-2-carbonyl)glycine ethyl ester were dissolved in 15 ml of 1-methyl-2-pyrrolidone; 0.841 ml of N-ethyldiisopropylamine was added, followed by stirring in an oil bath (90-100° C.) for 24 hours. After cooling, ice water were added, followed by extraction with ethyl acetate; the extract was washed with saturated brine, dried over magnesium sulfate and concentrated under reduced pressure. The residue was subjected to silica gel column chromatography and eluted with ethyl acetate:methanol:triethylamine (95:5:1). The desired fraction was collected and recrystallized from ethyl acetate to give 1.28 g of the title compound.

Melting point : 172-174° C. Elemental analysis: for C₃₂H₃₈N₆O₄.0.5H₂O. Calculated (%): C, 66.30; H, 6.78; N, 14.50. Found (%) : C, 66.42; H, 6.68,N, 14.55.

### Reference Example 8

### Production of ethyl 2-[6-[3-[4-(diphenylmethoxy)piperidino]propylamino]-3-methylimidazo[1,2-b]pyridazin-2-yl]-2-methylpro pionate dihydrochloride

2.38 g of 4-(diphenylmethoxy)-1-piperidinepropanamine and 1.03 g of ethyl 2-(6-chloro-3-methylimidazo[1,2-b]pyridazin-2-yl)-2-methylpropionate were stirred at 160° C. for 7.5 hours. After cooling, aqueous sodium bicarbonate was added, followed by extraction with ethyl acetate; the extract was washed with saturated brine, dried over magnesium sulfate and concentrated under reduced pressure. The residue was subjected to silica gel column chromatography and eluted with ethyl acetate:methanol:triethylamine (50:5:1). The desired fraction was collected, concentrated under reduced pressure and dissolved in 5 ml of ethyl acetate; 0.96 ml of a 4 N solution of hydrogen chloride in ethyl acetate was added, followed by concentration again. The residue was powdered by the addition of ethyl ether, collected by filtration and dried to give 666 mg of the title compound.

Amorphous

Elemental analysis: for C₃₄H₄₅N₅O₃Cl₂.1.5H₂O. Calculated (%): C, 60.98; H, 7.22; N, 10.46. Found (%) : C, 60.70; H, 6.95; N, 10.34.

### Assessment Example 1

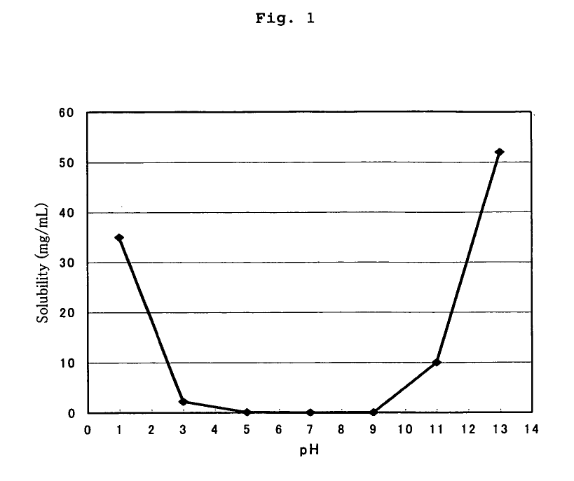

Regarding a compound A, the solubility was measured by measuring the solubility in the Britton-Robinson buffer at 20° C. at each pH of pHs 1, 3, 5, 7, 9, 11 and 13. The compound A was added to a solution at each pH, the mixture was vigorously stirred for 30 seconds every 5 minutes, and an amount of a physiologically active substance dissolved at a time point of 30 minutes was measured (according to a method of confirming the solubility described in Japanese Pharmacopoeia, general rules). It can be seen that, as shown in FIG. 1, the compound A is an amphoteric compound which is hardly dissolved in an aqueous solution at a neutral pH, and has the enhanced solubility on an acidic side or an alkaline side.

### Control Example 1

After a compound A (412.5 g), lactose (3465 g) and corn starch (612.2 g) were uniformly mixed in a fluidized-bed granulation drier, the mixture was granulated by spraying in the drier an aqueous solution in which hydroxypropylcellulose (HPC-L)(138.6 g) was dissolved, and then dried in the same drier. The resulting granule was ground using a power mill and the particle size was adjusted with a 1.5 mmφ punching screen. Further, 3871 g of this granule was taken, to this were added croscarmellose sodium (Ac-Di-Sol)(207 g) and magnesium stearate (62.1 g), and they were mixed by using a tumbler mixing machine to obtain a mixed granule. This mixed granule was compressed into a tablet having a weight of 300 mg using a 9.5 mmφ pestle with a tabletting machine to obtain a crude tablet.

### Example 1

After a compound A (825 g), lactose (2087 g) and corn starch (742.5 g) were uniformly mixed in a fluidized-bed granulation drier, the mixture was granulated by spraying in the drier an aqueous solution in which hydroxypropylcellulose (HPC-L)(148.5 g) was dissolved, and then dried in the same drier. The resulting granule was ground using a power mill and a 1.5 mmφ punching screen to obtain a granule of the compound A. 3181 g of this granule was taken, to this were added tartaric acid (690 g), croscarmellose sodium (Ac-Di-Sol)(207 g) and magnesium stearate (62.1 g), and they were mixed by using a tumbler mixing machine to obtain a mixed granule. This granule was compressed into a tablet having a weight of 300 mg using a 9.5 mmφ pestle with a tabletting machine to obtain a crude tablet.

### Example 2

After citric acid (anhydrous)(6100 g), crystalline cellulose (Avicel PH101)(2928 g) and colloidal anhydrous silica (122 g) were uniformly mixed, the powder was ground. 8850 g of this ground powder was taken, to this were added crystalline cellulose (Avicel PH302) (2832 g), polyvinylpyrrolidone (PVP-K30)(1708 g), lactose (5133 g) and magnesium stearate (177 g), and they were mixed by using a tumbler mixing machine. This powder was compressed into a tablet having a weight of about 300 mg using a 9.5 mmφ pestle with a tabletting machine to obtain a crude tablet. The resulting crude tablet was ground using a power mill and a 1.5 mmφ punching screen to obtain a citric acid granule. Separately, after a compound A (1649 g), lactose (2229 g) and corn starch (612.2 g) were uniformly mixed in a fluidized-bed granulation drier, the mixture was granulated by spraying in the drier an aqueous solution in which hydroxypropylcellulose (HPC-L)(138.6 g) was dissolved, and then dried in the same drier. The resulting granule was ground using a power mill and a particle size was adjusted with a 1.5 mmφ punching screen. 3647 g of this granule of the compound A was taken, to this were added the citric acid granule (3530 g), croscarmellose sodium (Ac-Di-Sol)(507 g) and magnesium stearate (117 g), and they were mixed by using a tumbler mixing machine to obtain a mixed granule. This mixed granule was compressed into a tablet having a weight of 300 mg using a 9.5 mmφ pestle with a tabletting machine to obtain a crude tablet.

### Example 3

After succinic acid (6100 g), crystalline cellulose (Avicel PH101)(2928 g) and colloidal anhydrous silica (122 g) were uniformly mixed, the powder was ground. To this ground powder (8850 g) were added crystalline cellulose (Avicel PH302)(2832 g), polyvinylpyrrolidone (PVP-K30)(1708 g), lactose (5133 g) and magnesium stearate (177 g), and they were mixed by using a tumbler mixing machine to obtain a mixed powder. This powder was compressed into a tablet having a weight of about 300 mg using a 9.5 mmφ pestle with a tabletting machine to obtain a crude tablet. The resulting crude tablet was ground using a power mill and a 1.5 mmφ punching screen to obtain a citric acid granule. Separately, after a compound A (1619 g), lactose (2229 g) and corn starch (612.2 g) were uniformly mixed in a fluidized-bed granulation drier, the mixture was granulated by spraying in the drier an aqueous solution hydroxypropylcellulose (HPC-L4 l(138.6 g) was dissolved, and then dried in the same drier. The resulting granule was ground using a power mill and a 1.5 mmφ punching screen to obtain a powder having an adjusted particle size. To this powder having an adjusted particle size (3647 g) were added an acid-mixed powder (3530 g), croscarmellose sodium (Ac-Di-Sol)(507 g) and magnesium stearate (117 g), and they were mixed by using a tumbler mixing machine to obtain a granule for compression. This granule was compressed into a tablet having a weight of 300 mg using a 9.5 mmφ pestle with a tabletting machine to obtain a crude tablet.

Table 1 shows formulations of Control Example 1, Example 1, Example 2 and Example 3 which contain the compound A.

TABLE 1

| | Control | | | |
|---|---|---|---|---|
| | Ex. 1* | Ex. 1 | Ex. 2 | Ex. 3 |
| Tartaric acid | — | 50.00 mg | — | — |
| Citric acid | — | — | 50.00 mg | — |
| (anhydrous) | | | | |
| Succinic acid | — | — | — | 50.00 mg |
| Avicel PH101 | — | — | 24.00 mg | 24.00 mg |
| Colloidal | — | — | 1.00 mg | 1.00 mg |
| anhydrous | | | | |
| silica | | | | |
| Lactose | — | — | 43.50 mg | 43.50 mg |
| Avicel PH302 | — | — | 24.00 mg | 24.00 mg |
| PVPK-30 | — | — | 6.00 mg | 6.00 mg |
| Magnesium | — | — | 1.50 mg | 1.50 mg |
| stearate | | | | |
| Compound A | 25.00 mg | 50.00 mg | 50.00 mg | 50.00 mg |
| Lactose | 210.0 mg | 126.50 mg | 67.50 mg | 67.50 mg |
| Corn starch | 37.10 mg | 45.00 mg | 18.55 mg | 18.55 mg |
| HPC-L | 8.40 mg | 9.00 mg | 4.20 mg | 4.20 mg |
| Ac-Di-Sol | 15.00 mg | 15.00 mg | 7.50 mg | 7.50 mg |
| Magnesium | 4.50 mg | 4.50 mg | 2.25 mg | 2.25 mg |
| stearate | | | | |
| Total | 300.00 mg | 300.00 mg | 300.00 mg | 300.00 mg |

*Two tablets were used in the confirmation of the absorbability.

### Assessment Example 2

As confirmation of the absorbability, a difference in an absorbing rate of a compound A due to a difference in a gastric pH was studied using a beagle dog. As a system having a low gastric pH, the dog was treated with histamine (30 μg/kg, s.c.) before administration of a preparation. Thereafter, histamine was administered s.c. every one hour, and a gastric pH was maintained in an acidic region. 15 minutes, 30 minutes, 1 hour, 2, 4 and 8 hours after administration of a preparation (50 mg/body), blood was taken with a heparin-treated syringe, the plasma was separated, and the blood concentration was measured. As a system having a high gastric pH, cimetidine (100 mg/body, twice a day) was orally administered from two days before administration of a preparation, and cimetidine (100 mg/kg) was intravenously administered 30 minutes before administration of a preparation. 15 minutes, 30 minutes, 1 hour, 2, 4 and 8 hours after administration of a preparation (50 mg/body), blood was taken with a heparin-treated syringe, the plasma was separated, and the blood concentration was measured.

The results of absorbability assessment are shown in Table 2. As apparent from the results, it was found that, even when the absorbability is lowered in a preparation of Control Example 1 containing no acidic compound, preparations of Examples 1, 2 and 3 containing an acidic compound are excellent in the absorbability, being a preparation having a small fluctuation in absorption.

TABLE 2

| | Under low gastric | | Under high gastric | |
|---|---|---|---|---|
| | pH condition | | pH condition | |
| | Cmax | AUC 0-8 hr | Cmax | AUC 0-8 hr |
| Sample | (μg/mL) | (hr · μg/mL) | (μg/mL) | (hr · μg/mL) |
| Control | 1.323 ± | 4.511 ± 0.815 | 0.061 ± 0.025 | 0.313 ± 0.144 |
| ex. 1 | 0.464 | | | |
| Ex. 1 | — | — | 1.070 ± 0.293 | 3.795 ± 0.862 |
| Ex. 2 | — | — | 0.962 ± 0.335 | 3.588 ± 1.121 |
| Ex. 3 | — | — | 0.557 ± 0.461 | 1.992 ± 1.547 |

Under low gastric pH condition: The condition in which gastric pH was adjusted to about pH 1.
Under high gastric pH condition: The condition in which gastric pH was adjusted to about pH 7.

### Example 4

Preparations were prepared in the formulation systems shown in Table 3. That is, for example, in the case of 100 mg tablet, a granule of a compound A (or diphenhydramine) comprising a compound A (or diphenhydramine)(1597 g), lactose (2163 g), corn starch (593.6 g) and hydroxypropylcellulose (HPC-L)(134.4 g) prepared by a fluidized-bed granulating method (FD-5S, POWLEX) and a citric acid granule comprising citric acid (anhydrous)(1600 g), crystalline cellulose (1152 g), sterile talc (192 g), colloidal anhydrous silica (32 g), lactose (1032 g), croscarmellose sodium (Ac-Di-Sol)(240 g) and magnesium stearate (96 g) prepared by dry granulation (Collect 12HUK, Kikusuiseisakusho) were mixed, and croscarmellose sodium (Ac-Di-Sol)(624 g) and magnesium stearate (144 g) were further added thereto to obtain a mixed granule. This mixed granule was compressed into a tablet using an oval type (8.0×14.0 mm) pestle with a tabletting machine (Collect 19K, Kikusuiseisakusho). To the resulting tablet was sprayed a film coating solution comprising hydroxypropylmethylcellulose (179.7 g), polyethylene glycol 6000 (36 g), titanium oxide (24 g) and red ferric oxide (0.32 g) using a pan type coating machine (Hicoater, Freund Sangyo), to obtain a film-coated tablet. Upon this, the condition was controlled so that a product temperature became 40° C. to 50° C. Similarly, a 12.5 mg tablet, a 25 mg tablet and a 50 mg tablet were prepared by adjusting the content of a compound A (or diphenhydramine) and the content of lactose in a granule.

TABLE 3

| | 12.5 mg | 25 mg | 50 mg | 100 mg |
|---|---|---|---|---|
| | tablet | tablet | tablet | tablet |
| Compound A | 12.5 mg | 25.0 mg | 50.0 mg | 100.0 mg |
| (or diphenhydramine) | | | | |
| Lactose | 287.0 mg | 274.5 mg | 249.5 mg | 199.5 mg |
| Corn starch | 37.1 mg | 37.1 mg | 37.1 mg | 37.1 mg |
| HPC-L | 8.4 mg | 8.4 mg | 8.4 mg | 8.4 mg |
| Citric acid | 100.0 mg | 100.0 mg | 100.0 mg | 100.0 mg |
| (anhydrous) | | | | |
| Crystalline cellulose | 72.0 mg | 72.0 mg | 72.0 mg | 72.0 mg |
| Colloidal anhydrous | 2.0 mg | 2.0 mg | 2.0 mg | 2.0 mg |
| silica | | | | |
| Sterile talc | 12.0 mg | 12.0 mg | 12.0 mg | 12.0 mg |
| Ac-Di-Sol | 54.0 mg | 54.0 mg | 54.0 mg | 54.0 mg |
| Magnesium stearate | 15.0 mg | 15.0 mg | 15.0 mg | 15.0 mg |
| Hydroxypropylmethyl | 11.2275 mg | 11.2275 mg | 11.2275 mg | 11.2275 mg |
| cellulose | | | | |
| Polyethylene glycol | 2.2500 mg | 2.2500 mg | 2.2500 mg | 2.2500 mg |
| 6000 | | | | |
| Titanium Oxide | 1.5000 mg | 1.5000 mg | 1.5000 mg | 1.5000 mg |
| Red ferric Oxide | 0.0225 mg | 0.0225 mg | 0.0225 mg | 0.0225 mg |
| Total | 615.0 mg | 615.0 mg | 615.0 mg | 615.0 mg |

### Assessment Example 3

Assessment of the stabilities of a 25 mg tablet of a compound A and a 100 mg tablet of a compound A prepared in Example 4 was performed by subdividing each 10 tablets of a preparation into a glass bottle, sealing the bottle, storing the bottle for 1 month in a system where the atmosphere was moisture-conditioned to 60% RH at 25° C. (relative humidity 60%) and in a system where the atmosphere was moisture-conditioned to 75% RH at 40° C. (relative humidity 75%), and observing and measuring the appearance, the content, the remaining ratio and the behavior of related substances. Confirmation of the resistance to light was performed by confirming the behavior of related substances regarding a sample of a tablet directly irradiated with 100,000 Luxes of a xenon lamp for 12 hours.

Table 4 shows the results of assessment of the stability of a 25 mg tablet of a compound A, and Table 5 shows the results of assessment of the stability of a 100 mg tablet of a compound A. As apparent from Table 4 and Table 5, a significant change in the nature, decrease in the content and remarkable production of related substances could not be seen, and the stability was better.

TABLE 4

| | | | The |
|---|---|---|---|
| Storage | | The indication | remaining |
| condition | Property | amount (%) | rate (%) |
| initial | Light pink | 99.9 | 100.0 |
| | film-coated | | |
| | tablets | | |
| 25° C. (sealed) | Light pink | 99.3 | 99.4 |
| 1 M, 60% RH | film-coated | | |
| | tablets | | |
| 40° C. (sealed) | Light pink | 97.9 | 97.9 |
| 1 M, 75% RH | film-coated | | |
| | tablets | | |
| Xenon lump | Light pink | — | — |
| 1,200,000 lux · hr | film-coated | | |
| | tablets | | |

TABLE 5

| | | The | The |
|---|---|---|---|
| | | indication | remaining |
| Storage condition | Property | amount (%) | rate (%) |
| initial | Light pink | 100.0 | 100.0 |
| | film-coated | | |
| | tablets | | |
| 25° C. (sealed) | Light pink | 99.6 | 99.6 |
| 1 M, 60% RH | film-coated | | |
| | tablets | | |
| 40° C. (sealed) | Light pink | 100.2 | 100.2 |
| 1 M, 75% RH | film-coated | | |
| | tablets | | |
| Xenon lump | Light pink | — | — |
| 1,200,000 lux · hr | film-coated | | |
| | tablets | | |

### Assessment Example 4

Each 10 tablets of a 12.5 mg tablet and a 100 mg tablet of a compound A prepared in Example 4 were subdivided into glass bottles, the bottles were sealed and stored for 1 month in a system where the atmosphere was moisture-conditioned to 60% RH at 25° C. (relative humidity 60%) and in a system where the atmosphere was moisture-conditioned to 75% RH at 40° C. (relative humidity 75%), respectively. In addition, regarding a 100 mg tablet of a compound (A), each 10 tablets were subdivided into glass bottles, and the bottles were also stored for 1 month in a system where the atmosphere was moisture-condition to 11% RH at 40° C. (relative humidity 11%) and in a system where the atmosphere was moisture-conditioned to 33% RH at 40° C. (relative humidity 33%), respectively, without closing the bottle.

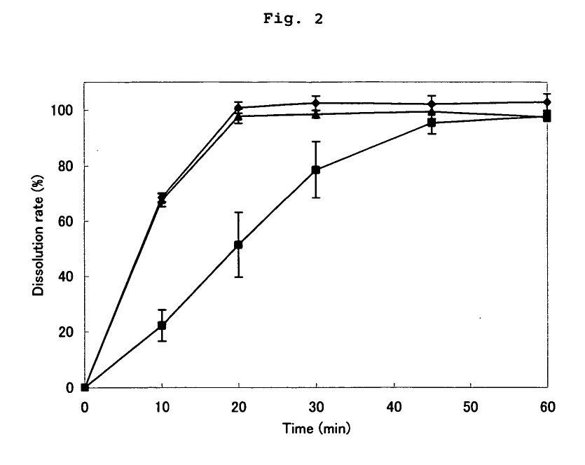

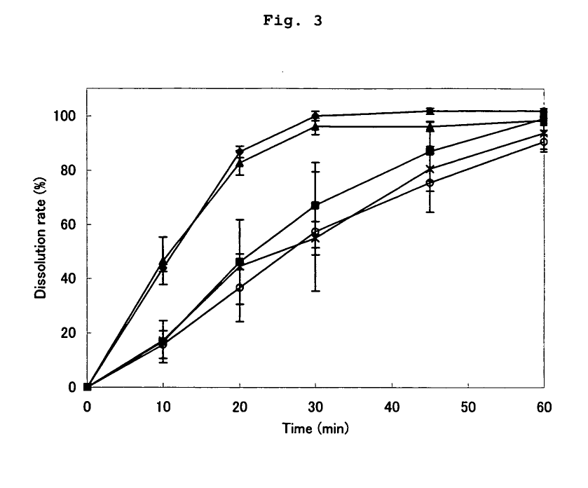

Regarding a tablet before storage and a tablet after storage, the dissolution behavior in an acetate buffer solution (900 mL) having pH of 3.8 at 37° C. and 50 rpm was confirmed (n=3), based on 14^{th} revision Japanese Pharmacopoeia dissolution test second method (paddle method). FIG. 2 shows a dissolution profile of a 12.5 mg tablet of a compound A prepared in Example 4, and FIG. 3 shows a dissolution profile of a 100 mg tablet of a compound A prepared in Example 4.

### Example 5

Preparations were prepared in formulation systems shown in Table 6. That is, for example, in the case of a 100 mg tablet, granules obtained by removing a 1000 μm or larger powder and a smaller than 150 μm powder from a granule of a compound A comprising a compound A (or diphenhydramine)(1597 g), lactose (2163 g), corn starch (593.6 g) and hydroxypropylcellulose (HPC-L)(134.4 g) prepared by a fluidized-bed granulating method (FD-5S, Powrex) and a citric acid granule comprising citric acid (anhydrous)(1600 g), crystalline cellulose (1152 g), sterile talc (192 g), colloidal anhydrous silica (32 g), lactose (1032 g), croscarmellose sodium (Ac-Di-Sol)(240 g) and magnesium stearate (96 g) prepared by dry granulation (Collect 12HUK, Kikusuiseisakusho) were mixed, and to this were added croscarmellose sodium (Ac-Di-Sol)(624 g) and magnesium stearate (144 g) to obtain a mixed granule. This mixed granule was compressed into a tablet with a tabletting machine (Collect 19K, Kikusuiseisakusho) using an oval type (8.0×14.0 mm) pestle. To the resulting tablet was sprayed a film coating solution comprising hydroxypropylmethylcellulose (179.7 g), polyethylene glycol 6000 (36 g), titanium oxide (24 g) and red ferric oxide (0.32 g) using a pan type coating machine (Hicoater, Freund Sangyo), to obtain a film-coated tablet. Upon this, the condition was controlled so that a product temperature became 40° C. to 50° C. Similarly, a 12.5 mg tablet, a 25 mg tablet and a 50 mg tablet were prepared by adjusting the content of a compound A (or diphenhydramine) and the content of lactose in a granule.

TABLE 6

| | 12.5 mg | 25 mg | 50 mg | 100 mg |
|---|---|---|---|---|
| | tablet | tablet | tablet | tablet |
| Compound A | 12.5 mg | 25.0 mg | 50.0 mg | 100.0 mg |
| (or diphenhyd- | | | | |
| ramine) | | | | |
| Lactose | 287.0 mg | 274.5 mg | 249.5 mg | 199.5 mg |
| Corn starch | 37.1 mg | 37.1 mg | 37.1 mg | 37.1 mg |
| HPC-L | 8.4 mg | 8.4 mg | 8.4 mg | 8.4 mg |
| Citric acid | 100.0 mg | 100.0 mg | 100.0 mg | 100.0 mg |
| (anhydrous) | | | | |
| Crystalline | 72.0 mg | 72.0 mg | 72.0 mg | 72.0 mg |
| cellulose | | | | |
| Colloidal | 2.0 mg | 2.0 mg | 2.0 mg | 2.0 mg |
| anhydrous | | | | |
| silica | | | | |
| Sterile talc | 12.0 mg | 12.0 mg | 12.0 mg | 12.0 mg |
| Ac-Di-Sol | 54.0 mg | 54.0 mg | 54.0 mg | 54.0 mg |
| Magnesium | 15.0 mg | 15.0 mg | 15.0 mg | 15.0 mg |
| stearate | | | | |
| Hydroxy- | 11.2275 mg | 11.2275 mg | 11.2275 mg | 11.2275 mg |
| propylmethyl | | | | |
| cellulose | | | | |
| Poly- | 2.2500 mg | 2.2500 mg | 2.2500 mg | 2.2500 mg |
| ethylene 6000 | | | | |
| Titanium oxide | 1.5000 mg | 1.5000 mg | 1.5000 mg | 1.5000 mg |
| Red ferric | 0.0225 mg | 0.0225 mg | 0.0225 mg | 0.0225 mg |
| oxide | | | | |
| Total | 615.0 mg | 615.0 mg | 615.0 mg | 615.0 mg |

### Assessment Example 5

Assessment of the stabilities of preparations prepared in Example 5 was performed by subdividing each 10 tablets of a preparation into a glass bottle, sealing the bottle, storing the bottle for 1 month in a system where the atmosphere was moisture-conditioned to 75% RH at 40° C. (relative humidity 75%), and observing and measuring the appearance, the content, the remaining ratio and the behavior of related substances.

Table 7 shows the results of stability assessment of a 12.5 mg tablet of a compound A prepared in Example 5, Table 8 shows the results of stability assessment of a 25 mg tablet of a compound A prepared in Example 5, Table 9 shows the results of the stability assessment of a 50 mg tablet of a compound A, and Table 10 shows stability assessment of a 100 mg tablet of a compound A prepared in Example 5. As apparent from Table 7 to Table 10, a significant change in the nature, decrease in the content and remarkable production of related substances could not be seen, and the stability was better.

TABLE 7

| | | The | The |
|---|---|---|---|
| Storage | | indication | remaining |
| condition | Property | amount (%) | rate (%) |
| initial | Light pink | 104.1 | 100.0 |
| | film-coated tablets | | |
| 40° C. (sealed) | Light pink | 103.5 | 99.4 |
| 1 M, 75% RH | film-coated tablets | | |

TABLE 8

| | | The | The |
|---|---|---|---|
| Storage | | indication | remaining |
| condition | Property | amount (%) | rate (%) |
| initial | Light pink | 99.6 | 100.0 |
| | film-coated tablets | | |
| 40° C. (sealed) | Light pink | 99.1 | 99.5 |
| 1 M, 75% RH | film-coated tablets | | |

TABLE 9

| | | The | The |
|---|---|---|---|
| Storage | | indication | remaining |
| condition | Property | amount (%) | rate (%) |
| initial | Light pink | 98.6 | 100.0 |
| | film-coated tablets | | |
| 40° C. (sealed) | Light pink | 97.6 | 99.0 |
| 1 M, 75% RH | film-coated tablets | | |

TABLE 10

| | | The | The |
|---|---|---|---|
| Storage | | indication | remaining |
| condition | Property | amount (%) | rate (%) |
| initial | Light pink | 98.6 | 100.0 |
| | film-coated tablets | | |
| 40° C. (sealed) | Light pink | 99.5 | 100.9 |
| 1 M, 75% RH | film-coated tablets | | |

### Assessment Example 6

Each 10 tablets of a 12.5 mg tablet and a 100 mg tablet of a compound A prepared in Example 5 were-subdivided into a glass bottle, and the bottle was sealed and stored for 1 month in a system where the atmosphere was moisture-conditioned to 75% RH at 40° C. (relative humidity 75%).

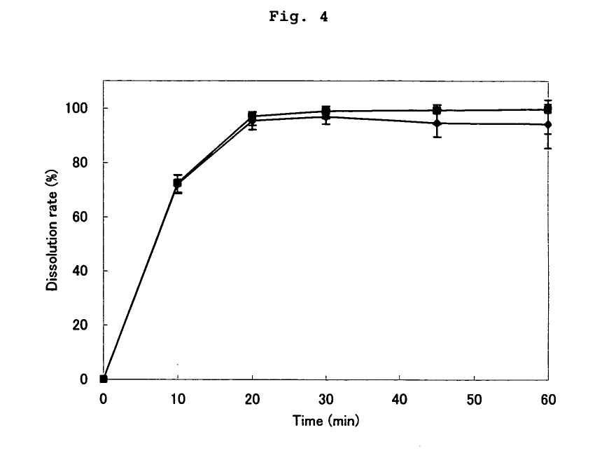

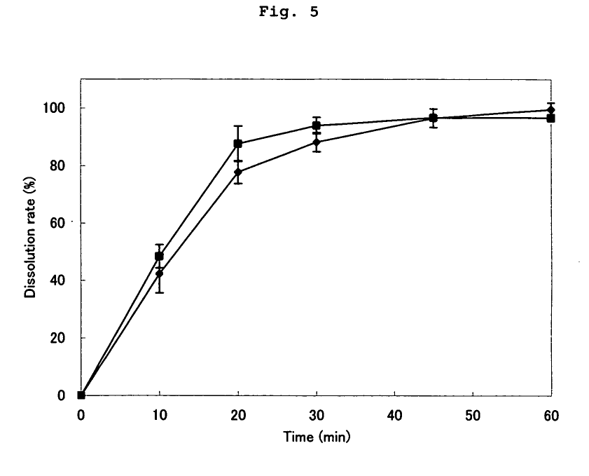

Regarding a tablet before storage and a tablet after storage, the dissolution behavior in an acetate buffer solution (900 mL) having pH of 3.8 at 37° C. and 50 rpm was conformed (n=6), based on 14^{th} revision Japanese Pharmacopoeia dissolution test second method (paddle method). FIG. 4 shows a dissolution profile of a 12.5 mg tablet of a compound A prepared in Example 5, and FIG. 5 shows a dissolution profile of a 100 mg tablet of a compound A prepared in Example 5.

It was found that a preparation of a compound A prepared in Example 5 has a smaller change in a dissolution profile as compared with a preparation of a compound A prepared in Example 4.

### Example 6

Preparations were prepared in formulation systems shown in Table 11. That is, for example, in the case of a 50 mg tablet, granules obtained by removing a 1000 μm or larger powder and a smaller than 150 μm powder from a granule of a compound A (or diphenhydramine) comprising a compound A (or diphenhydramine)(1000 g), lactose (1350 g), cornstarch (371.0 g) and hydroxypropylcellulose (HPC-L)(84.0 g) prepared by a fluidized-bed granulating method (FD-5S, Powrex) and a citric acid granule comprising citric acid (anhydrous)(1000 g), crystalline cellulose (480 g), sterile talc (120 g), colloidal anhydrous silica (20 g), lactose (645 g), croscarmellose sodium (Ac-Di-Sol)(150 g) and magnesium stearate (60 g) prepared by dry granulation (roller compactor, Alexanderwerk) were mixed, and to this were further added croscarmellose sodium (Ac-Di-Sol)(390 g), crystalline cellulose (240 g) and magnesium stearate (60 g), to obtain a mixed granule. This mixed granule was compressed into a tablet with a tabletting machine (Collect 19K, Kikusuiseisakusho) using a 9.5 mmφ pestle. To the resulting tablet was sprayed a film coating solution comprising hydroxypropylmethylcellulose (336.825 g), polyethylene glycol 6000 (67.5 g), titanium oxide (45 g) and red ferric oxide (0.675 g) using a pan type coating machine (Hicoater, Freund Sangyo), to obtain a film-coated tablet. Upon this, the condition was controlled so that a product temperature became 40° C. to 50° C. Similarly, a 12.5 mg tablet and a 25 mg tablet were prepared by adjusting the content of a compound A (or diphenhydramine) and the content of lactose in a granule.

TABLE 11

| | | 12.5 mg | 25 mg | 50 mg |
|---|---|---|---|---|
| | placebo | tablet | tablet | tablet |
| Compound A | 0 mg | 12.5 mg | 25.0 mg | 50.0 mg |
| (or diphenhydramine) | | | | |
| Lactose | 102.5 mg | 105.0 mg | 92.5 mg | 67.5 mg |
| Avicel PH101 (placebo) | 15.0 mg | 0 mg | 0 mg | 0 mg |
| Corn starch | 18.55 mg | 18.55 mg | 18.55 mg | 18.55 mg |
| HPC-L | 4.2 mg | 4.2 mg | 4.2 mg | 4.2 mg |
| Citric acid (anhydrous) | 50.0 mg | 50.0 mg | 50.0 mg | 50.0 mg |
| Avicel PH101 | 24.0 mg | 24.0 mg | 24.0 mg | 24.0 mg |
| Colloidal anhydrous | 1.0 mg | 1.0 mg | 1.0 mg | 1.0 mg |
| silica | | | | |
| Granulated lactose | 32.25 mg | 32.25 mg | 32.25 mg | 32.25 mg |
| Avicel PH302 | 12.0 mg | 12.0 mg | 12.0 mg | 12.0 mg |
| Sterile talc | 6.0 mg | 6.0 mg | 6.0 mg | 6.0 mg |
| Ac-Di-Sol | 27.0 mg | 27.0 mg | 27.0 mg | 27.0 mg |
| Magnesium stearate | 7.5 mg | 7.5 mg | 7.5 mg | 7.5 mg |
| Hydroxypropylmethyl | 7.485 mg | 7.485 mg | 7.485 mg | 7.485 mg |
| cellulose | | | | |
| Polyethylene 6000 | 1.500 mg | 1.500 mg | 1.500 mg | 1.500 mg |
| Titanium oxide | 1.000 mg | 1.000 mg | 1.000 mg | 1.000 mg |
| Red ferric oxide | 0.015 mg | 0.015 mg | 0.015 mg | 0.015 mg |
| Total | 310.0 mg | 310.0 mg | 310.0 mg | 310.0 mg |

### Assessment Example 7

Assessment of the stability of preparations prepared in Example 6 was performed by subdividing each 10 tablets of a preparation into a glass bottle, sealing the bottle, storing the bottle for 1 month in a system where the atmosphere was moisture-conditioned to 75% RH at 40° C. (relative humidity 75%), and observing and measuring the appearance, the content, the remaining ratio and the behavior of related substances.

Table 12 shows the results of stability assessment of a 12.5 mg tablet of a compound A prepared in Example 6, Table 13 shows the results of stability assessment of a 25-mg tablet of a compound A prepared in Example 6, and Table 14 shows stability assessment of a 50 mg tablet of a compound A prepared in Example 6. As apparent from Table 12 to Table 14, a significant change in the nature, decrease in the content and remarkable production of related substances could not be seen, and the stability was better.

TABLE 12

| | | The | The |
|---|---|---|---|
| Storage | | indication | remaining |
| condition | Property | amount (%) | rate (%) |
| initial | Light pink film- | 97.3 | 100.0 |
| | coated tablets | | |
| 40° C. (sealed) | Light pink film- | 97.5 | 100.2 |
| 1 M, 75% RH | coated tablets | | |

TABLE 13

| | | The | The |
|---|---|---|---|
| Storage | | indication | remaining |
| condition | Property | amount (%) | rate (%) |
| initial | Light pink film- | 96.0 | 100.0 |
| | coated tablets | | |
| 40° C. (sealed) | Light pink film- | 99.1 | 103.2 |
| 1 M, 75% RH | coated tablets | | |

TABLE 14

| | | The | The |
|---|---|---|---|
| Storage | | indication | remaining |
| condition | Property | amount (%) | rate (%) |
| initial | Light pink film- | 98.2 | 100.0 |
| | coated tablets | | |
| 40° C. (sealed) | Light pink film- | 101.7 | 103.6 |
| 1 M, 75% RH | coated tablets | | |

### Assessment Example 8

Each 10 tablets of a 12.5 mg tablet, a 25 mg tablet and a 50 mg tablet of a compound A prepared in Example 6 were subdivided into a glass bottle, and the bottle was sealed and stored for 1 month in a system where the atmosphere was moisture-conditioned to 75% RH at 40° C. (relative humidity 75%).

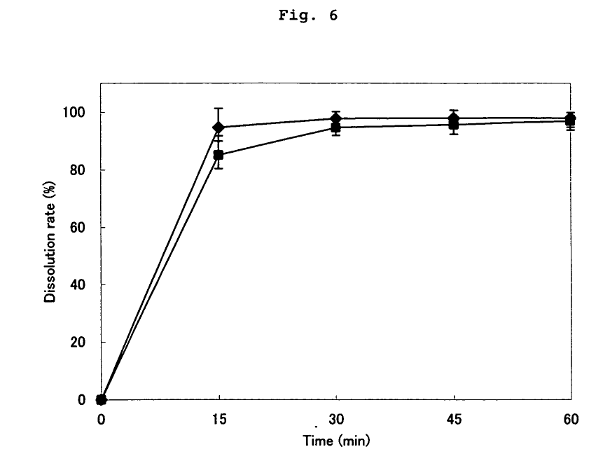

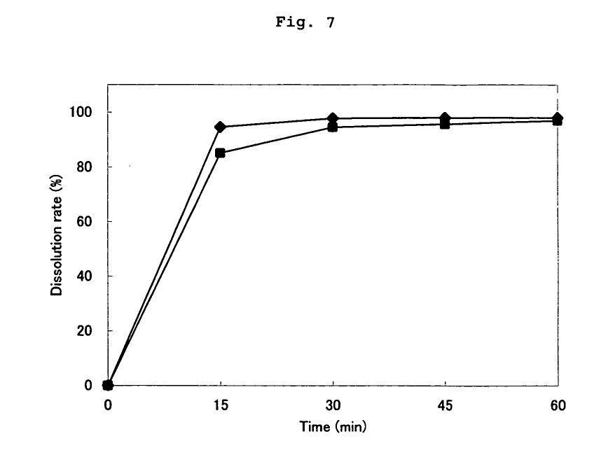

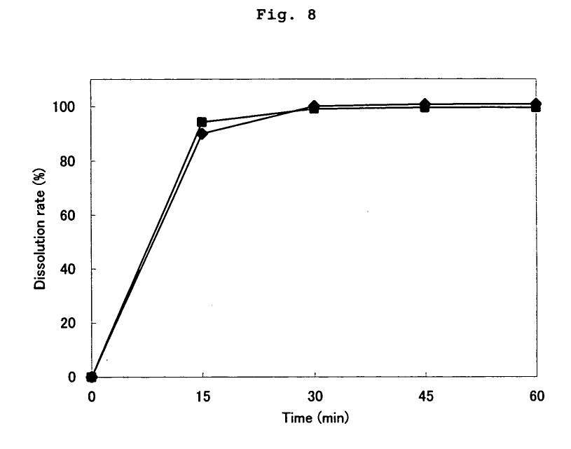

Regarding a tablet before storage and a tablet after storage, the dissolution behavior in an acetate buffer solution (900 mL) having pH of 3.8 at 37° C. and 50 rpm was confirmed (n=6), based on 14^{th} revision Japanese Pharmacopoeia dissolution test second method (paddle method). FIG. 6 shows a dissolution profile of a 12.5 mg tablet of a compound A prepared in Example 6, FIG. 7 shows a dissolution profile of a 25 mg tablet of a compound A prepared in Example 6, and FIG. 8 shows a dissolution profile of a 50 mg tablet of a compound A prepared in Example 6.

It was found that a preparation of a compound A prepared in Example 6 has a smaller change in a dissolution profile as compared with a preparation of a compound A prepared in Example 4.

Industrial Applicability

The acid-blended preparation of the present invention remarkably improves the digestive tract absorbability of a physiologically active substance, and is excellent in the stability.

1. A preparation comprising blending (I) a compound having the anti-allergy activity, the anti-histamine activity, the anti-inflammatory activity, the anti-PAF activity and/or the eosinophile chemotaxis inhibiting activity, and (2) an acidic compound.

2. The preparation as claimed in claim 1, wherein the compound having the anti-allergy activity, the anti-histamine activity, the anti-inflammatory activity, the anti-PAF activity and/or the eosinophile chemotaxis inhibiting activity is a basic compound.

3. The preparation as claimed in claim 1, wherein the compound having the anti-allergy activity, the anti-histamine activity, the anti-inflammatory activity, the anti-PAF activity and/or the eosinophile chemotaxis inhibiting activity is an amphoteric compound.

4. The preparation as claimed in claim 1, wherein the solubility of the compound having the anti-allergy activity, the anti-histamine activity, the anti-inflammatory activity, the anti-PAF activity and/or the eosinophile chemotaxis inhibiting activity at pH 3 or lower is 10 times or more the solubility at pH 5 to 8.

5. The preparation as claimed in claim 1, wherein the compound having the anti-allergy activity, the anti-histamine activity, the anti-inflammatory activity, the anti-PAF activity and/or the eosinophile chemotaxis inhibiting activity is a compound represented by the formula: 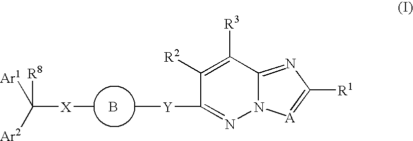
wherein Ar¹ and Ar² each is an aromatic group optionally having substituents, Ar¹ and Ar² optionally form a condensed cyclic group together with the adjacent carbon atom, ring B is a nitrogen-containing heterocycle optionally having substituents, X and Y are the same or different and each is a bond, an oxygen atom, S(O)ₚ (wherein p is an integer of 0 to 2), NR⁴(wherein R⁴ is a hydrogen atom or a lower alkyl group) or a divalent linear lower hydrocarbon group optionally having substituents and containing 1 to 3 hetero atom(s), A is a nitrogen atom or CR⁷ (wherein R⁷ is a hydrogen atom, a halogen atom, a hydrocarbon group optionally having substituents, an acyl group or a hydroxy group optionally having substituents), R¹, R² and R³ are the same or different and each is a hydrogen atom, a halogen atom, a hydrocarbon group optionally having substituents, an acyl group or a hydroxy group optionally having substituents, R⁸ is a hydrogen atom, a hydroxy group optionally substituted by a lower alkyl group, or a carboxyl group or a salt thereof.

6. The preparation as claimed in claim 1, wherein the compound having the anti-allergy activity, anti-histamine activity, the anti-inflammatory activity, the anti-PAF activity and/or the eosinophile chemotaxis inhibiting activity is ethyl
2-[6-[3-[4-(diphenylmethoxy)piperidino]propylamino]imidazo[1,2-b]pyridazin-2-yl]-2-methylpropionate,
2-[6-[3-[4-(diphenylmethoxy)piperidino]propylamino]imidazo[1,2-b]pyridazin-2-yl]-2-methylpropionic acid or a salt thereof,
N-[6-[3-[4-(diphenylmethoxy)piperidino]propylamino]imidazo[1,2-b]pyridazin-2-carbonyl]glycine ethyl ester or a salt thereof, ethyl
2-[6-[3-[4-(diphenylmethoxy)piperidino]propylamino]-3-methylim idazo[1,2-b]pyridazin-2-yl]-2-methylpropionate or a salt thereof, or
2-[6-[3-[4-(diphenylmethoxy)piperidino]propylamino]imidazo[1,2-b]pyridazin-2-yl]-2-methylpropionic acid dihydrate.

7. The preparation as claimed in claim 1, wherein the acidic compound is solid.

8. The preparation as claimed in claim 1, wherein 50% or more of particles constituting the acidic compound are particles of 50 μm to 1.5 mm.

9. The preparation as claimed in claim 1, wherein 50% or more of particles constituting the acidic compound are particles of 150 μm to 1.0 mm.

10. The preparation as claimed in claim 1, wherein particles of 50 μm or smaller among particles constituting the acidic compound are 20% or less of the all particles.

11. The preparation as claimed in claim 1, wherein the acidic compound is carboxylic acid, sulfonic acid, acidic polysaccharide or acidic amino acid.

12. The preparation as claimed in claim 1, wherein the acidic compound is carboxylic acid.

13. The preparation as claimed in claim 12, wherein carboxylic acid is fumaric acid, adipic acid, malic acid, acetic acid, tartaric acid, succinic acid or citric-acid.

14. The preparation as claimed in claim 12, wherein carboxylic acid is tartaric acid, succinic acid or citric acid.

15. The preparation as claimed in claim 12, wherein carboxylic acid is citric acid.

16. The preparation as claimed in claim 1, which contains 0.1 to 10 parts by weight of the acidic compound relative to 1 part by weight of the compound having the anti-allergy activity, the anti-histamine activity, the anti-inflammatory activity, the anti-PAF activity and/or the eosinophile chemotaxis inhibiting activity.

17. The preparation as claimed in claim 1, which is a tablet.

18. The preparation as claimed in claim 1, which comprises blending a granule containing the compound having the anti-allergy activity, the anti-histamine activity, the anti-inflammatory activity, the anti-PAF activity and/or the eosinophile chemotaxis inhibiting activity and a granule containing the acidic compound.

19. The preparation as claimed in claim 18, wherein the granule containing the compound having the anti-allergy activity, the anti-histamine activity, the anti-inflammatory activity, the anti-PAF activity and/or the eosinophile chemotaxis inhibiting activity contains 50% or more particles of 50 μm to 1.5 mm, and the granule containing the acidic compound contains 50% or more of particles of 50 μm to 1.5 mm.

20. The preparation as claimed in claim 18, wherein the granule containing the compound having the anti-allergy activity, the anti-histamine activity, the anti-inflammatory activity, the anti-PAF activity and/or the eosinophile chemotaxis inhibiting activity contains 50% or more particles of 150 μm to 1.0 mm, and the granule containing the acidic compound contains 50% or more of particles of 150 μm to 1.0 mm.

21. The preparation as claimed in claim 1, which is a multi-layered tablet.

22. The preparation as claimed in claim 17 or claim 21, which is a coated preparation.

23. The preparation as claimed in claim 1, wherein talc and/or magnesium stearate is (are) further added thereto.

24. A process for preparing the preparation as claimed in claim 1, which comprises incorporating a granule containing a compound having the anti-allergy activity, the anti-histamine activity, the anti-inflammatory activity, the anti-PAF activity and/or the eosinophile chemotaxis inhibiting activity and a granule containing an acidic compound.
